(19) United States
(12) Patent Application Publication
(10) Pub. No.: US 2015/0099800 A1
(43) Pub. Date: Apr. 9, 2015

(54) NOVEL LIPASE INHIBITORS, REPORTER SUBSTRATES AND USES THEREOF

(71) Applicant: Northeastern University, Boston, MA (US)

(72) Inventor: Richard I. Duclos, JR., Quincy, MA (US)

(21) Appl. No.: 14/403,441

(22) PCT Filed: May 24, 2013
(86) PCT No.: PCT/US13/42600

§ 371 Date: Nov. 24, 2014

Related U.S. Application Data
(60) Provisional application No. 61/651,245, filed on May 24, 2012.

(51) Int. Cl. C07D 305/12; G01N 21/76; C07D 263/06; G01N 30/90; C07C 229/18; C07D 271/12

(52) U.S. Cl. CPC C07D 305/12; C07C 229/18; C07D 271/12; C07D 263/06; G01N 30/90; G01N 21/76
USPC 514/449; 549/263; 560/22; 548/126; 548/300.1; 435/19; 422/44

(57) ABSTRACT

The invention provides for novel lipase inhibitors, and compositions and devices comprising the same. The invention further provides for methods for treatment of disorders comprising administration of novel diacylglycerol lipase inhibitors, and compositions and devices comprising said inhibitors. In some embodiments, the disorders are pancreatitis, obesity, shock or pancreatic necrosis. The invention further provides for novel ether lipid reporter compounds and methods of assaying enzymatic activity comprising contacting a compound with a novel ether lipid reporter compound.

This application claims the benefit of and priority to U.S. Provisional Patent Application No. 61/651,245, filed on May 24, 2012, the content of which is hereby incorporated by reference in its entirety.

### GOVERNMENT SUPPORT

This invention was made with government support under NIDA Grant Nos. R03-DA-24842 and T32-DA-07312; and under DOE Grant No. DE-SC0005251. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

The catalytic sites of diacylglycerol lipases (DAGLs) are unique among the lipases and hydrolases in effecting selectivity for the hydrolysis of the 1-acyl group of 1,2-diacyl-sn-glycerol substrates (J. Cell Biol. 2003, 163, 463; herein incorporated by reference in its entirety). The DAGLα (120 KDa) and DAGLβ (70 KDa) isoforms are both present during brain development where their presynaptic axonal location suggests an important role in this period of neuritogenesis, rapid cell growth, and plasticity (J. Cell Biol. 2003, 163, 463; Biochem. Biophys. Res. Commun. 2011, 411, 809; Trends Pharmacol. Sci. 2007, 28, 83; J. Neuroendocrinol. 2008, 20 (Suppl. 1), 75; J. Neurosci. Res. 2010, 88, 735; each herein incorporated by reference in its entirety). However, the ultimate postsynaptic dendritic location of the α-isoform in the adult brain is consistent with the subsequent role of DAGLα in endocannabinoid paracrine retrograde signaling with a lesser role for the DAGLβ isoform (J. Cell Biol. 2003, 163, 463; J. Neurosci. Res. 2010, 88, 735; J. Neurosci. 2006, 26, 4740; J. Neurosci. 2006, 26, 5628; Mol. Pharmacol. 2007, 72, 612; J. Neurosci. 2007, 27, 3663; J. Neurosci. 2008, 28, 2976; Neuropharmacology 2008, 54, 95; J. Neurosci. 2010, 30, 2017; Neuron 2010, 65, 320; each herein incorporated by reference in its entirety). Thus, DAGLs have an important role in the endocannabinoid system as they are primarily responsible for releasing endocannabinoid 2-arachidonoylglycerol (2-AG) from diacylglycerols, including 1-stearoyl-2-arachidonoyl-sn-glycerol that is the principal 1,2-diacyl-sn-glycerol component of brain and nerves, for signaling at cannabinoid receptors (Biochem. Biophys. Acta 1972, 270, 337; Adv. Exp. Med. Biol. 1992, 318, 413; Nicolaou, A; Kokotos, G. Bioactive Lipids, Nicolaou, A; Kokotos, G.; The Oily Press: Bridgwater, UK, 2004; pp 294; Biochem. Biophys. Res. Comm. 1995, 215, 89; Prostaglandins, Leukotrienes Essent. Fatty Acids 2002, 66, 173; Nat. Rev. Neurosci. 2003, 4, 873; Pharmacol. Biochem. Behav. 2005, 81, 224; Prog. Lipid Res. 2006, 45, 405; J. Physiol. 2007, 584, 373; Trends Biochem. Sci. 2007, 32, 27; Neuropharmacology 2008, 54, 58; each herein incorporated by reference in its entirety).

There is a need for novel diacylglycerol lipase inhibitors. There is also a need for novel treatments for a variety of disease states for which diacylglycerol lipase is implicated. There is a further need for novel fluorescent resonance energy transfer reporter substrates for lipase assays.

### SUMMARY OF THE INVENTION

In one aspect, the invention is directed to a compound of formula (I)

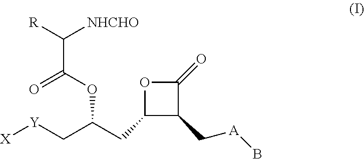

wherein
R is (C₁-C₁₂)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is a solid support or H;
X is a solid support or H;
Y is a linking group comprising -J-, —O-J-, —S-J-, —N(H)-J-, or —N((C₁-C₃)-alkyl)-J-;
J is (C₁-C₁₂)-alkyl or (OCH₂CH₂)ₙ—, wherein any carbon atom in said (C₁-C₁₂)-alkyl or (OCH₂CH₂)ₙ— is optionally replaced with one or more heteroatom, cycloalkyl group, heterocycle, aryl or heteroaryl group; and

each n is independently 0-100; or a pharmaceutically acceptable salt thereof.

In some embodiments, at least one of B or X is a solid support.

In another aspect, the invention is directed to compositions comprising a compound of formula (I) and a pharmaceutically acceptable carrier.

In another aspect, the invention is directed to a device comprising:
a) a compound of formula (I)

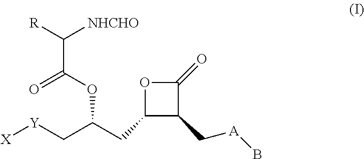

wherein
R is (C₁-C₁₂)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or (OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is a solid support or H;
X is a solid support or H, wherein at least one of B or X is a solid support;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100; or a pharmaceutically acceptable salt thereof;
b) a first conduit configured to deliver blood of a subject to contact the compound of formula (I); and
c) a second conduit configured to return blood to the subject.

In another aspect, the invention is directed to a method of inhibiting diacylglycerol lipase comprising contacting diacylglycerol lipase with a compound of formula (I) or a composition comprising a compound of formula (I).

In another aspect, the invention is directed to a method of treating a disorder in a subject in need thereof comprising administration of a therapeutically effective amount of a compound of formula (I). In some embodiments, the disorder is pancreatitis. In some embodiments, the disorder is obesity.

In another aspect, the invention is directed to a method of treating a disorder in a subject in need thereof comprising contacting the blood of the subject with a compound of formula (I). In some embodiments, the compound of formula (I) is attached to a solid support. In some embodiments, the disorder is pancreatitis. In some embodiments, the disorder is obesity.

In another aspect, the invention is directed to an inhibitor of protease activity, wherein said inhibitor has the formula X—Y—Z, wherein X is H or a solid support; Y is a linking group comprising alkyl, polyethyleneglycol or a combination thereof; and Z is 6-amidino-2-naphthyl 4-guanadinobenzoate, benzamidine, leupeptin or another inhibitor of protease activity. In some embodiments, Z is an inhibitor of phospholipase A₂ activity.

In another aspect, the invention is directed to a method of treating a disorder in a subject in need thereof comprising contacting the blood of the subject with a compound of formula X—Y—Z. In some embodiments, the compound of formula X—Y—Z is attached to a solid support. In some embodiments, the disorder is pancreatitis. In some embodiments, the disorder is obesity.

In another aspect, the invention is directed to a method of treating pancreatitis comprising contacting the blood of a subject over a solid-supported inhibitor of lipase, or proteases, or phospholipase A2, or any combination thereof, passing the blood of the patient over the solid-supported inhibitor with any device that then returns the blood to the patient. In some embodiments, the lipase inhibitor is a compound of formula (I). In some embodiments, the lipase inhibitor is a compound of formula X—Y—Z.

In another aspect, the invention is directed to a method of treating pancreatitis comprising orally administering a non-absorbable form of a lipase inhibitor on a polymeric support. In some embodiments, the lipase inhibitor is a compound of formula (I). In some embodiments, the lipase inhibitor is a compound of formula X—Y—Z.

In another aspect, the invention is directed to a method of treating shock comprising orally administering a non-absorbable form of a lipase inhibitor on a polymeric support. In some embodiments, the lipase inhibitor is a compound of formula (I). In some embodiments, the lipase inhibitor is a compound of formula X—Y—Z.

In another aspect, the invention is directed to a method of treating shock comprising contacting the blood of a subject over a solid-supported inhibitor of lipase, or proteases, or phospholipase A2, or any combination thereof, passing the blood of the patient over the solid-supported inhibitor with any device that then returns the blood to the patient. In some embodiments, the lipase inhibitor is a compound of formula (I). In some embodiments, the lipase inhibitor is a compound of formula X—Y—Z.

In another aspect, the invention is directed to a method of treating pancreatic necrosis comprising orally administering a non-absorbable form of a lipase inhibitor on a polymeric support. In some embodiments, the lipase inhibitor is a compound of formula (I). In some embodiments, the lipase inhibitor is a compound of formula X—Y—Z.

In another aspect, the invention is directed to a method of treating pancreatic necrosis comprising contacting the blood of a subject over a solid-supported inhibitor of lipase, or proteases, or phospholipase A2, or any combination thereof, passing the blood of the patient over the solid-supported inhibitor with any device that then returns the blood to the patient. In some embodiments, the lipase inhibitor is a compound of formula (I). In some embodiments, the lipase inhibitor is a compound of formula X—Y—Z.

In some embodiments, the solid support comprises a surface-modified polyvinyl tubing having reduced plasticizer leaching and improved physical properties for tubing with medical applications involving extended contact with blood and other tissue, such as in transfusions, organ bypass surgeries, kidney dialysis, platelet donation, medical drains, oral intubation, and others.

In another aspect, the invention is directed to a compound of formula (II)

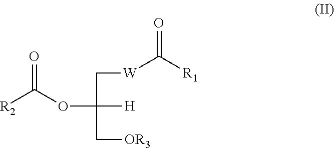

wherein
W is O, NH, or N—(C₁-C₃)-alkyl;
R₁ is (C₁-C₁₂)-alkyl; (C₁-C₁₂)-alkyl-aryl, wherein aryl is optionally substituted with one or more nitro groups; (C₁-C₁₂)-alkyl-NH-aryl, wherein aryl is optionally substituted with one or more nitro groups; —NH(C₁-C₈)-alkyl, —O(C₁-C₈)-alkyl, —NH(C₁-C₈)-alkyl,

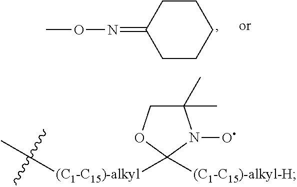

R₂ is (C₁-C₂₀)-alkyl; (C₁-C₂₀)-alkenyl; (C₁-C₂₀)-alkyl-aryl, wherein aryl is optionally substituted with one or more nitro groups; (C₁-C₂₀)-alkyl-NH-aryl, wherein aryl is optionally substituted with one or more nitro groups; (C₁-C₂₀)-alkyl-heteroaryl, wherein heteroaryl is optionally substituted with one or more nitro groups; or —NH(C₁-C₈)-alkyl; and
R₃ is H or (C₁-C₁₂)-alkyl; or a pharmaceutically acceptable salt thereof.

In some embodiments of formula (II), W is O, NH, or N—(C₁-C₃)-alkyl;

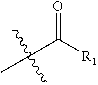

is 4-pyrenebutyryl, 10-pyrenedecanoyl, 5-doxylstearoyl, 16-doxylstearoyl, or dinitrophenyl-∈-amino-n-caproyl;

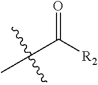

is 4-pyrenebutyryl, dinitrophenyl-∈-amino-n-caproyl, or 6-(N-(7-nitrobenz-2-oxa-1,3-diazol-4-yl)amino)hexanoyl; and

R₃ is H or (C₁-C₁₂)-alkyl; or a pharmaceutically acceptable salt thereof.

In another aspect, the invention is directed to a method of assaying DAGL activity comprising contacting a compound with a compound of formula (II).

Still other objects and advantages of the invention will become apparent to those of skill in the art from the disclosure herein, which is simply illustrative and not restrictive. Thus, other embodiments will be recognized by the skilled artisan without departing from the spirit and scope of the invention.

### BRIEF DESCRIPTION OF THE FIGURES

FIG. 1 shows various inhibitors of DAGL activity.

FIG. 2 shows reporter substrates for in vitro FRET-based DAGL and related lipase assays.

FIG. 3 shows structures and names of compounds referred to herein.

FIG. 4 shows ether lipid analogs of RHC80267 (1) and O-3841 (2).

FIG. 5 shows a Western blot of hDAGLα and mDAGLβ proteins. hDAGLα and mDAGLβ cDNA sequences were both subcloned into the pcDNA3.1-V5-HIS-TOPO vector. The corresponding plasmids and mock (empty pcDNA3.1) were transfected into HEK293T according to the manufacturer's protocol and cell lysates analyzed via PAGE. Western blot with anti-V5 was used to confirm expression.

FIG. 6 shows purity of [¹⁴C]SAG substrate by TLC with phosphorimaging analysis. A) [1″-¹⁴C]1-Stearoyl-2-arachidonoyl-sn-glycerol ([¹⁴C]SAG) 20,000 DPM eluted (4:96 acetone/chloroform) on TLC (silica gel) with a 1 h exposure. B) [1″-¹⁴C]1-Stearoyl-2-arachidonoyl-sn-glycerol ([¹⁴C]SAG) 9,400 DPM eluted (4:96 acetone/chloroform) on TLC (boric acid treated with silica gel plate, Rf=0.21) with a 15 h exposure. C) the profile of the laine with less than 0.5% 1(3)-diglyceride rearrangement byproduct [1′″-¹⁴C]1-stearoyl-3-arachidonoyl-sn-glycerol (Rf=0.42) present upon quantitative phosphorimaging analysis.

FIG. 7 shows radio-TLC assay data for inhibitors (3)-(8) (100 nM and 10 nM) of mDAGLα.

FIG. 8 shows DMSO solubilizes FRET reporter substrate (17) for hDAGLα hydrolysis. Cell lysate of hDAGLα expression with reporter compound (17) shows the effect of DMSO to solubilize the lipid substrate. No increase in activity was observed at DMSO concentrations above 10% (data not shown). The same effect on solubilization of substrate with DMSO for lipoprotein lipase was also observed (data not shown).

FIG. 9 shows evaluation of FRET reporter substrates (17)-(22) with hDAGLα. Reporter compounds as substrates for hDAGLα activity with HEK293T cell lysates in the presence of 10% DMSO are shown. A) Pyrenyl analogs (17)-(19), (21), and (22) (EX 320 nm, EM 400 nm). B) NBD analog (20) (EX 485 nm, EM 535 nm).

FIG. 10 shows inhibition of hDAGLα activity by THL (3) in the FRET assay with reporter (17).

### DETAILED DESCRIPTION OF THE INVENTION

The catalytic sites of diacylglycerol lipases (DAGLs) are unique among the lipases and hydrolases in effecting selectivity for the hydrolysis of the 1-acyl group of 1,2-diacyl-sn-glycerol substrates (J. Cell Biol. 2003, 163, 463; herein incorporated by reference in its entirety). The DAGLα (120 KDa) and DAGLβ (70 KDa) isoforms are both present during brain development where their presynaptic axonal location suggests an important role in this period of neuritogenesis, rapid cell growth, and plasticity (J. Cell Biol. 2003, 163, 463; Biochem. Biophys. Res. Commun. 2011, 411, 809; Trends Pharmacol. Sci. 2007, 28, 83; J. Neuroendocrinol. 2008, 20 (Suppl. 1), 75; J. Neurosci. Res. 2010, 88, 735; each herein incorporated by reference in its entirety). However, the ultimate postsynaptic dendritic location of the α-isoform in the adult brain is consistent with the subsequent role of DAGLα in endocannabinoid paracrine retrograde signaling with a lesser role for tile DAGLβ isoform (J. Cell Biol. 2003, 163, 463; J. Neurosci. Res. 2010, 88, 735; J. Neurosci. 2006, 26, 4740; J. Neurosci. 2006, 26, 5628; Mol. Pharmacol. 2007, 72, 612; J. Neurosci. 2007, 27, 3663; J. Neurosci. 2008, 28, 2976; Neuropharmacology 2008, 54, 95; J. Neurosci. 2010, 30, 2017; Neuron 2010, 65, 320; each herein incorporated by reference in its entirety). Thus, DAGLs have an important role in the endocannabinoid system as they are primarily responsible for releasing endocannabinoid 2-arachidonoylglycerol (2-AG) from diacylglycerols, including 1-stearoyl-2-arachidonoyl-sn-glycerol that is the principal 1,2-diacyl-sn-glycerol component of brain and nerves, for signaling at cannabinoid receptors (Biochem. Biophys. Acta 1972, 270, 337; Adv. Exp. Med. Biol. 1992, 318, 413; Nicolaou, A; Kokotos, G. Bioactive Lipids, Nicolaou, A; Kokotos, G.; The Oily Press: Bridgwater, UK, 2004; pp 294; Biochem. Biophys. Res. Comm. 1995, 215, 89; Prostaglandins, Leukotrienes Essent. Fatty Acids 2002, 66, 173; Nat. Rev. Neurosci. 2003, 4, 873; Pharmacol. Biochem. Behav. 2005, 81, 224; Prog. Lipid Res. 2006, 45, 405; J. Physiol. 2007, 584, 373; Trends Biochem. Sci. 2007, 32, 27; Neuropharmacology 2008, 54, 58; each herein incorporated by reference in its entirety). Small molecules that inhibit DAGL would have major effects on lipid metabolism. For example, a lower rate of DAGL-catalyzed biosynthesis of endocannabinoid 2-arachidonoylglycerol (2-AG) may reduce 2-AG activation of cannabinoid receptors. The attenuation of signaling by the constitutive cannabinoid receptors would be distinct from the effects of inverse-agonist drugs binding to the cannabinoid receptors and may be of pharmacological utility.

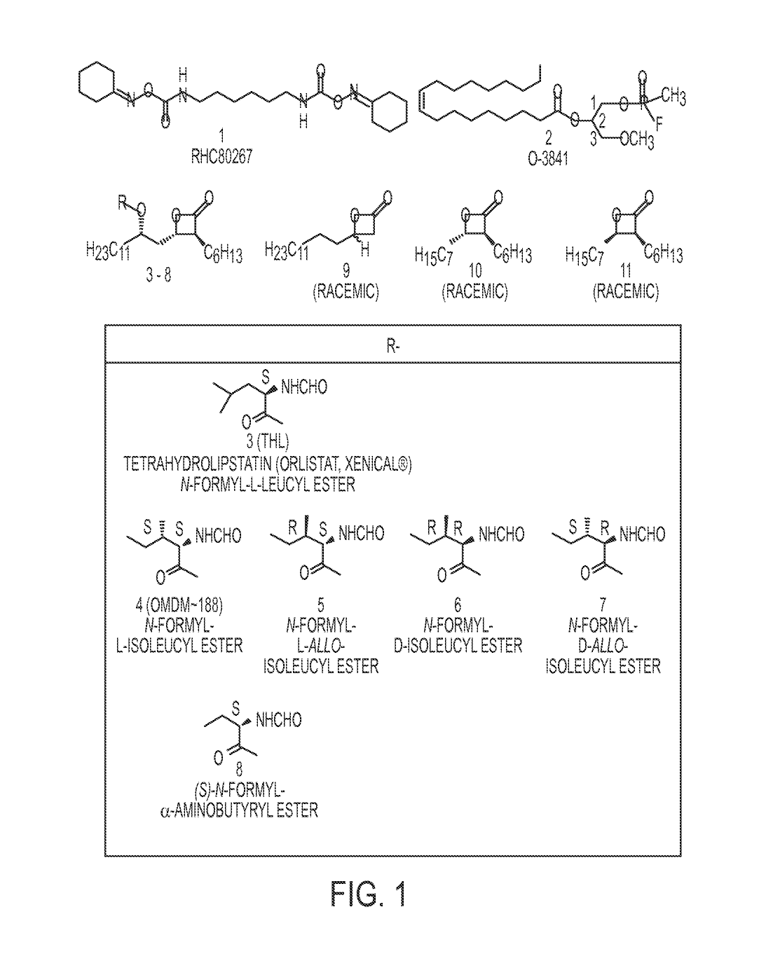

Exemplary compound (I) inhibitors of DAGL activity are shown in FIG. 1, along with known inhibitors (1)-(4).

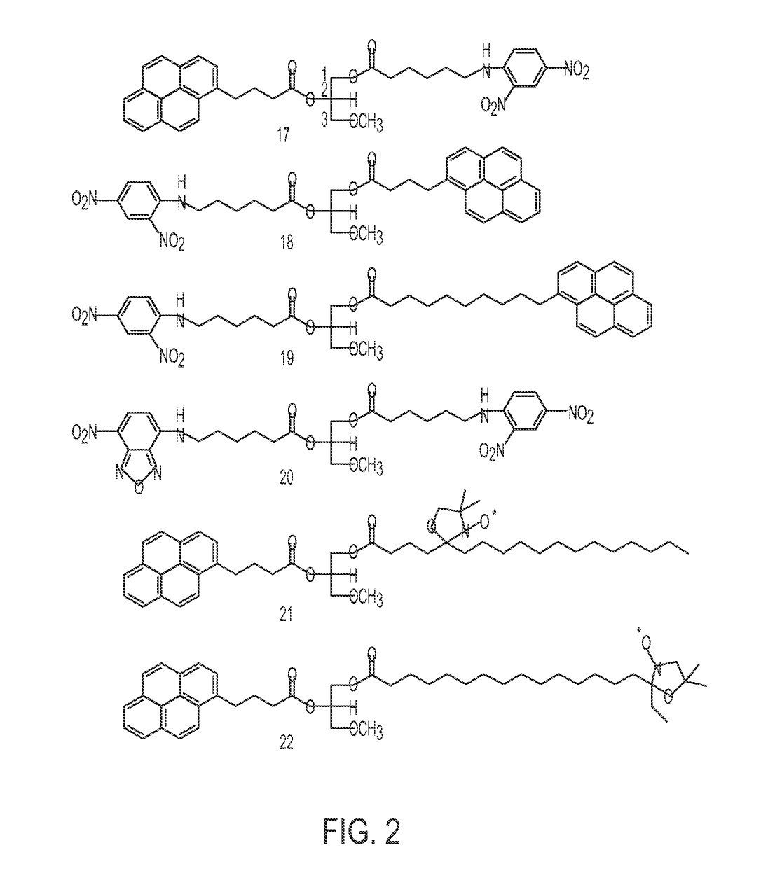

Exemplary compound (II) fluorescent resonance energy transfer (FRET) reporter substrates for in vitro assays of DAGL activity are shown in FIG. 2.

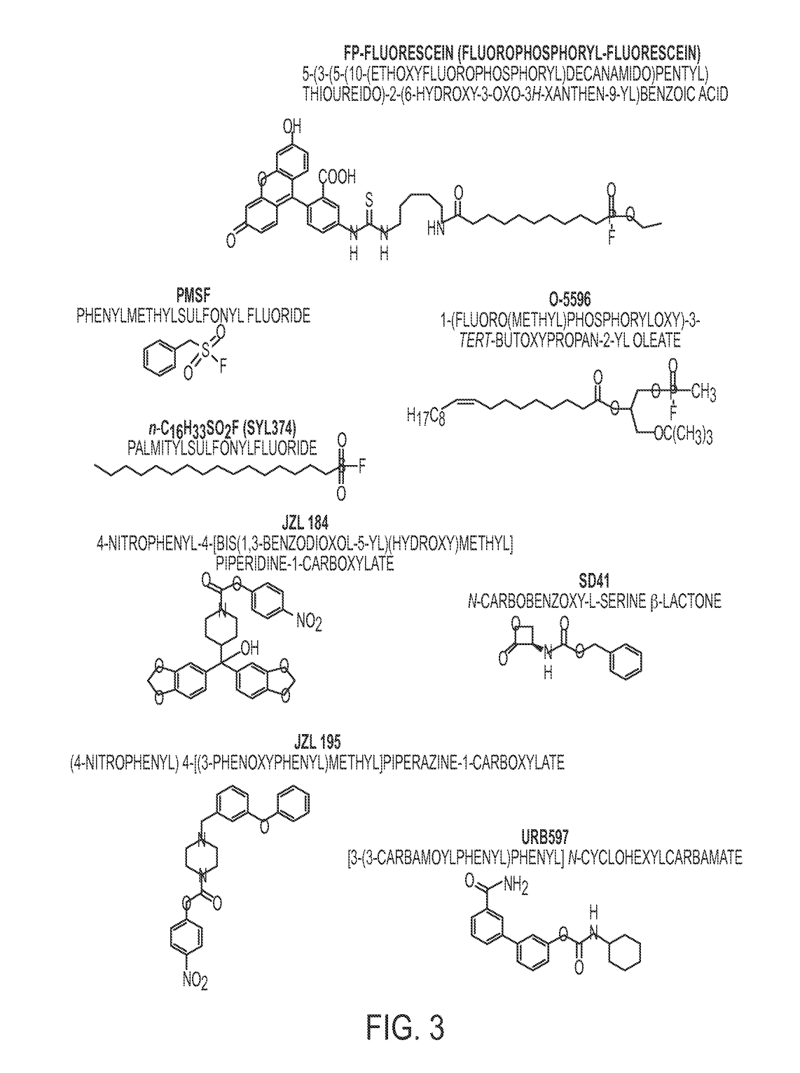

Four known inhibitors of DAGL activity include bis-oximinocarbamate RHC80267 1 (J. Cell Biol. 2003, 163, 463; J. Physiol. 2006, 577, 263; each herein incorporated by reference in its entirety), fluorophosphonate O-3841 2 (Biochim. Biophys. Acta 2006, 1761, 205; herein incorporated by reference in its entirety), and tetrahydrolipstatin (3, THL) (J. Cell Biol. 2003, 163, 463; Bioorg. Med. Chem. Lett. 2008, 18, 5838; each herein incorporated by reference in its entirety); and the N-formyl-L-isoleucyl ester OMDM-188 4 (Ortar, G.; Bisogno, T.; Ligresti, A.; Morera, E.; Nalli, M.; Di Marzo, V. J. Med. Chem. 2008, 51, 6970; each herein incorporated by reference in its entirety) (FIG. 1) that inhibit hDAGLα with apparent IC50 values of 65,000 nM, 160 nM, and 60 nM, respectively. The transition-state-mimicking fluorophosphonate group (RP=OOEtF) of FP-fluorescein utilized for the identification of serine hydrolases does not react appreciably with DAGL, unlike fluorophosphonate O-3841 (ROP=OMeF) and its t-butyl analog O-5596 (FIG. 3) (Biochim. Biophys. Acta 2006, 1761, 205; Bioorg. Med. Chem. Lett. 2008, 18, 5838; ChemMedChem 2009, 4, 946; each herein incorporated by reference in its entirety). Also, DAGL activity is not affected by phenylmethylsulfonylfluoride (PhCH₂SO₂F) (J. Cell Biol. 2003, 163, 463; herein incorporated by reference in its entirety). There have been several previously reported analytical methodologies for measuring DAGL activities including radio-TLC, LC-MS, and the use of general esterase reporter molecules (J. Cell Biol. 2003, 163, 463; Biochim. Biophys. Acta 2006, 1761, 205; Methods Enzymol. 1982, 86, 11; Toxicol. Appl. Pharmacol. 2001, 173, 48; each herein incorporated by reference in its entirety).

The compounds of formula (I) can be used to treat various disorders. Included in these disorders are pancreatitis and obesity. Acute pancreatitis has an incidence of approximately 123,600 cases per year in the United States, approximately 15% (18,500) of which proceed to a severe condition with approximately 30% (5,560) leading to death (Granger and Remick, (2005) Shock. 24 Suppl 1:45-51; herein incorporated by reference in its entirety). Acute pancreatitis is most dangerous for obese patients. Removing pancreatic lipase and lipoprotein lipase from the blood should attenuate the risk of multi-organ failures. Pancreatitis is discussed in, for example, Pancreatology 2008, 8, 257-264; Shock 2004, 22(5), 467-471; Gasteroenterology 1989, 97, 1521-1526; and Sci. Transl. Med. 2013, 5, 169ra11; each herein incorporated by reference in its entirety.

The digestive enzymes of the pancreas include lipase (hydrolyzes fat), secreted phospholipase A₂ (sPLA₂ hydrolyzes the sn-2-acyl groups of phospholipids), amylase (hydrolyzes starch), and enzymes for the digestion of dietary protein including trypsin (cleaves at lysine and arginine residues) and chymotrypsin (cleaves at phenylalanine, tyrosine, and tryptophan residues). The release of these active enzymes (in some cases ultimately from their zymogen or proenzyme forms) into the pancreas, bloodstream and intraperitoneal space occurs in the early stages of acute necrotizing pancreatitis (ANP). The most dangerous component is the pancreatic lipase, with some potential roles for pancreatic serine proteases and phospholipase A₂. Especially at the site of pancreatic necrosis and in the bloodstream, the lipase hydrolyzes fat while the phospholipase A₂ hydrolyzes phospholipids. Cytotoxic levels of free fatty acids are released that are responsible for inflammatory responses that can result in multiple organ failures (lung, kidney, others) (Sci. Transl. Med. 2011, 3, 107ra110; Am. J. Physiol. Heart Circ. Physiol. 2008, 294:H1779-H1792; and J. Proteome Res. 2013, 12, 347-362; each herein incorporated by reference in its entirety). The risk of death from acute pancreatitis is particularly high for obese individuals.

The related condition of shock can occur from ischemic intestinal necrosis due to pancreatic secretions in combination with intestinal disfunction resulting from trauma, sepsis, burns, and radiation. See, for example, Amer. J. Physiol. Heart Circ. Physiol 2008, 294, H1779; Shock 2004, 22, 467; Microcirculation 2005, 12, 71; Sci. Trans. Med. 2013, 5, 169ra1; each herein incorporated by reference in its entirety.

The role of nonesterified fatty acids in necrotizing pancreatitis has been studied in rodents in vivo (Sci. Transl. Med. 2011, 3, 107ra110; herein incorporated by reference in its entirety). Lipotoxicity was clearly demonstrated in these studies, but administered lipase inhibitor tetrahydrolipstatin (THL) was not effective in preventing pancreatic and extrapancreatic fat necrosis (Gastroenterology 1992, 103, 1916; Pancreas 2001, 23, 341; each; herein incorporated by reference in its entirety). The intraperitoneal (IP) injections of lipase inhibitor tetrahydrolipstatin (THL) had a small effect in lowering lipase activity in the blood, but no effect on the concentration of free fatty acid in the blood, which is generally highly regulated by the liver (Pancreas 2001, 23, 341; herein incorporated by reference in its entirety).

In another aspect, the invention comprises a medical device that returns blood to the patient after the blood is exposed to a compound of formula (I). The medical device for use with the compound of formula (I) will reduce lipase activity in the blood and especially at the site of injury attenuating the release of inflammatory lipids, thus reducing the risk of multiple organ failures and the resulting late-stage deaths associated with pancreatitis. The device is not expected to be effective on any pro-inflammatory lipids that enter the bloodstream via the lymph from peritoneal fluid (ascites) (Am. J. Surgery 2012, 203, 211; herein incorporated by reference in its entirety). However, a lipase protein in ascites that enters the bloodstream via the lymph will be removed by the device.

In another aspect, a device is provided comprising
a) a compound of formula (I)
b) a first conduit configured to deliver blood of a subject to contact the compound of formula (I); and
c) a second conduit configured to return blood to the subject.

In some embodiments, the solid support comprises a glass slide, a polymer bead, plastic tubing, glass tubing, rubber tubing. In some embodiments, the solid support comprises medical grade polyvinyl chloride tubing.

In some embodiments, the first and/or second conduit comprises plastic tubing, glass tubing or rubber tubing. In some embodiments, the first and/or second conduit comprises medical grade polyvinyl chloride tubing. The conduits can be made of any material that is compatible with blood of a subject, and may be further attached to a pump to enable routing blood through the conduits.

In the necrotized pancreas without intact blood vessels, pancreatic enzymes enter the blood stream. Pancreatic lipase (along with lipoprotein lipase, serum esterases, and related enzymes with lipase activity) will be removed from blood by the solid-supported lipase inhibitor device, and peripheral effects of free fatty acid will be blocked. More importantly, lipase damage to the pancreas and local fat will be reduced as the lipase is not recirculated, but diffuses into the blood and is removed by covalent bonding to the device. Blood levels of lipase activity can be readily monitored to determine when the course of treatment is complete. The progression of pancreatitis can be monitored by following blood pancreatic amylase levels coupled with appropriate radiology studies.

In some embodiments, the invention provides methods of treating cancer comprising administering the solid supported compound of formula (I) to a subject in need thereof. In some embodiments, the invention provides methods of treating cancer comprising contacting the blood of a subject in need thereof with a compound of formula (I). Methods of treating blood to remove cancer cells using devices such as nanoparticles or microfluidics are discussed, for example, in Chemical & Engineering News 2013, 91(6), 31 and Chemical & Engineering News 2013, 91(15), 28-29; each herein incorporated by reference in its entirety.

In some embodiments, the medical device delivers blood to the solid-supported compound of formula (I), rather than administering greasy drug(s) to the aqueous biological environment where transport and metabolism are not well controlled. This device comprises solid-supported inhibitors of lipase, or proteases, or phospholipase A₂, or any combination thereof, attached via linker(s) to, for example, polyvinyl chloride tubing, or in any device that allows passing the blood of the patient over the solid-supported form of the drug (or drugs) and the blood returned to the patient with the corresponding lipase, protease, or phospholipase activity attenuated. Analogously, such a device for the handling of blood has been proposed to treat bacterial sepsis (Lee, J.-J.; Jeong, K. J.; Hashimoto, M.; Kwon, A. H.; Rwei, A.; Shankarappa, S. A.; Tsui, J. H.; Kohane, D. S, Nano Lett. 2013, 13, ASAP; herein incorporated by reference in its entirety).

The novel surface-modified polyvinyl chloride tubing will have reduced plasticizer leaching and improved physical properties for intravenous use. The device in the form of surface-modified tubing will be sterilized by, for example, gamma irradiation methods currently used for single-use medical equipment.

Alternatively, in the second immobilized-drug-form of the device, solid supported (non-absorbable) drugs (inhibitors of lipase, or proteases, or phospholipase A₂, or any combination) are delivered directly and exclusively through the gastrointestinal tract and reach the small intestinal site of damage from secreted juices of a diseased (pancreatitis, shock, or others) pancreas. Unlike the non-solid-supported enzyme treatments shown to be effective in treating shock at the lumen of the intestine, the immobilized-drug-form of the device is not absorbed and reaches preferably to the critical treatment location.

In certain embodiments, the blood treatment and gastrointestinal drug forms of the invention avoid systemic distribution of drugs. Most dosage concerns can be avoided. Immunological responses to this therapy are minimized. There are currently very few medical therapies for acute necrotizing pancreatitis and shock.

Accordingly, methods of treating such disorders are disclosed. These methods comprise administering a therapeutically effective amount of at least one of the compounds of this disclosure, or a pharmaceutically acceptable salt thereof, to a subject in need thereof, thereby treating the disorder. In some embodiments, the blood of a subject can be passed through the solid-supported form of the compound to decrease lipase activity, the blood being returned to the patient by any method or device, thereby treating the disorder. In some embodiments, a subject in need of treatment can be one afflicted with one or more of the disorders described herein.

A subject can be a mammal including, but not limited to, a human, a monkey, such as a cynomolgous monkey, a chimpanzee, a bird, a farm animal (e.g., a cow, goat, horse, pig, or sheep), a pet (e.g., a cat, dog, or guinea pig, rat, or mouse), or laboratory animal (e.g., an animal model for a disorder). Non-limiting representative subjects can be a human infant, a pre-adolescent child, an adolescent, an adult, or a senior/elderly adult. In some embodiments, the subject is a mouse, rat or human. In some embodiments, the subject is a mouse. In some embodiments, the subject is a rat. In some embodiments, the subject is a human.

### ABBREVIATIONS AND DEFINITIONS

The term “alkyl” as used herein means a saturated linear, branched or cyclic alkyl group having from 1 to about 20 carbon atoms, and advantageously 1 to about 7 carbon atoms including, for example, methyl, ethyl, propyl, butyl, hexyl, octyl, isopropyl, isobutyl, sec-butyl, tert-butyl, cyclopropyl, cyclohexyl, and cyclooctyl.

The term “alkenyl” as used herein means a straight or branched hydrocarbon chain containing about 2 to 20 carbons and containing at least one carbon-carbon double bond. Representative alkenyl groups include ethenyl, 2-propenyl, 2-methyl-2-propenyl, 2-methylhex-2-enyl, 3-butenyl, 4-pentenyl, 5-hexenyl, 2-heptenyl, 2-methyl-1-heptenyl, and 3-decenyl.

The term “alkynyl” as used herein means a straight or branched chain hydrocarbon group containing about 2 to 20 carbon atoms and containing at least one carbon-carbon triple bond. Representative alkynyl groups include acetylenyl, 1-propynyl, 2-propynyl, 3-butynyl, 2-pentynyl, and 1-butynyl.

The term “aryl” as used herein means an aromatic ring system that includes only carbon as ring atoms, for example phenyl, biphenyl or naphthyl. The aryl group can be unsubstituted, singly substituted or, if possible, multiply substituted, with substituent groups in any possible position.

The term “heteroaryl” as used herein means an unsaturated ring structure having about 5 to about 20 ring members comprising carbon atoms and one or more heteroatoms, including oxygen, nitrogen and/or sulfur. Exemplary heteroaryl groups include thiophene, oxazole, isoxazole, imidazole, pyrazole, benzimidazole, triazolopyridine, benzotriazole, pyridine, pyridine 1-oxide, pyrimidine, indole, indazole, furan, quinoline, 1,2,4-triazole, 1,2,3-triazole, imidazole, and tetrazole. The heteroaromatic ring can be unsubstituted, singly substituted or, if possible, multiply substituted, with substituent groups in any possible position.

The term “solid support” as used herein means a biocompatible material, for example medical grade PVC or other derivatizable polymer, that is compatible with contacting blood. Other exemplary solid supports are discussed herein, and include alkoxy polyethyleneglycols, alkoxy polyethyleneglycol halides, celluloses, cellulose halides, or other biocompatible non-absorbable polymer.

The term “linking group” as used herein means a substituted or unsubstituted alkyl group, alkenyl group or alkynyl group, a polyethylene glycol group, alcohols, aryl groups, heteroaryl groups (for example triazoles and tetrazoles), heterocyclic groups and carbocyclic groups. Other exemplary linking groups are discussed herein.

The term “compound of the invention” as used herein means a compound of formula (I) or (II), or any subgenus or species thereof. The term is also intended to encompass salts, hydrates, and solvates thereof.

The term “composition(s) of the invention” as used herein means compositions comprising a compound of the invention. The compositions of the invention may further comprise other agents such as, for example, carriers, excipients, stabilants, lubricants, solvents, and the like.

A “pharmaceutical composition” refers to a mixture of one or more of the compounds of the invention with other chemical components, such as physiologically acceptable carriers and excipients. The purpose of a pharmaceutical composition is to facilitate administration of a compound to an organism or subject.

The term “pharmaceutically acceptable salt” is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids; and salts derived from inorganic or organic bases including, for example sodium, potassium, calcium, ammonium or tetrafluoroborate. Exemplary pharmaceutically acceptable salts are found, for example, in Berge, et al. (J. Pharm. Sci. 1977, 66(1), 1; hereby incorporated by reference in its entirety).

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in Remington's Pharmaceutical Sciences (Alfonso Gennaro ed., Krieger Publishing Company (1997); Remington's: The Science and Practice of Pharmacy, 21^{st} Ed. (Lippincot, Williams & Wilkins (2005); Modern Pharmaceutics, vol. 121 (Gilbert Banker and Christopher Rhodes, CRC Press (2002); each of which hereby incorporated by reference in its entirety).

As used herein, the term “effective amount” refers to an amount of a compound disclosed herein, which is sufficient to reduce or ameliorate the severity, duration, progression, or onset of a disease or disorder. The precise amount of compound administered to a subject will depend on the mode of administration, the type and severity of the disease or condition and on the characteristics of the subject, such as general health, age, sex, body weight and tolerance to drugs. The skilled artisan will be able to determine appropriate dosages depending on these and other factors. When co-administered with other agents, e.g., when co-administered with an anti-cancer agent, an “effective amount” of the second agent will depend on the type of drug used. Suitable dosages are known for approved agents and can be adjusted by the skilled artisan according to the condition of the subject, the type of condition(s) being treated and the amount of a compound being used. In cases where no amount is expressly noted, an effective amount should be assumed.

Herein, novel inhibitors of DAGL activity are reported that generally resemble diglycerides. DAGL proteins used in these studies are also reported, as are assay conditions using a radiolabeled endogenous diglyceride substrate.

In some embodiments, R is (C₁-C₆)-alkyl or (C₆-C₁₂)-aryl. In some embodiments, R is (C₁-C₄)-alkyl or (C₆)-aryl. In some embodiments, R is (C₂-C₄)-alkyl or phenyl. In some embodiments, R is (C₂-C₄)-alkyl. In some embodiments, R is ethyl or sec-butyl.

In some embodiments, A is —V—, —V—O—, —V—S—, —V—N(H)—, or —V—N((C₁-C₃)-alkyl)-. In some embodiments, A is —V—, —V—O—, —V—S—, or —V—N(H)—. In some embodiments, A is —V. In some embodiments, A is —V—O—. In some embodiments, A is —V—S—. In some embodiments, A is —V—N(H)—. In some embodiments, A is —V—N((C₁-C₃)-alkyl)-.

In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, heterocycle, aryl or heteroaryl group. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, aryl or heteroaryl group. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more aryl or heteroaryl group. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more aryl group. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroaryl group.

In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one heteroatom, heterocycle, aryl or heteroaryl group. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one heteroatom. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one aryl or heteroaryl group. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one aryl group. In some embodiments, V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one heteroaryl group. In some embodiments, V is (C₁-C₁₂)-alkyl. In some embodiments, V is —(OCH₂CH₂).

In some embodiments, V is (C₁-C₁₂)-alkyl, wherein any carbon atom is optionally replaced with one heteroatom, heterocycle, aryl or heteroaryl group. In some embodiments, V is (C₁-C₁₂)-alkyl, wherein any carbon atom is optionally replaced with one heteroatom, aryl or heteroaryl group. In some embodiments, V is (C₁-C₁₂)-alkyl, wherein any carbon atom is optionally replaced with one heteroatom. In some embodiments, V is (C₁-C₁₂)-alkyl, wherein any carbon atom is optionally replaced with one aryl group. In some embodiments, V is (C₁-C₁₂)-alkyl, wherein any carbon atom is optionally replaced with one heteroaryl group.

In some embodiments, Y is -J-, —O-J-, —S-J-, —N(H)-J-, or —N((C₁-C₃)-alkyl)-J-. In some embodiments, Y is —O-J-, —S-J-, or —N(H)-J-. In some embodiments, Y is -J-. In some embodiments, Y is —O-J-. In some embodiments, Y is —S-J-. In some embodiments, Y is —N(H)-J-. In some embodiments, Y is —N((C₁-C₃)-alkyl)-J-.

In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, heterocycle, aryl or heteroaryl group. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, aryl or heteroaryl group. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more aryl or heteroaryl group. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more aryl group. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroaryl group.

In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one heteroatom, heterocycle, aryl or heteroaryl group. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one heteroatom. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one aryl or heteroaryl group. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one aryl group. In some embodiments, J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one heteroaryl group. In some embodiments, J is (C₁-C₁₂)-alkyl. In some embodiments, J is —(OCH₂CH₂)ₙ—.

In some embodiments, J is (C₁-C₁₂)-alkyl, wherein any carbon atom is optionally replaced with one heteroatom, heterocycle, aryl or heteroaryl group. In some embodiments, J is (C₁-C₁₂)-alkyl, wherein any carbon atom is optionally replaced with one heteroatom, aryl or heteroaryl group. In some embodiments, J is (C₁-C₁₂)-alkyl, wherein any carbon atom is optionally replaced with one heteroatom. In some embodiments, J is (C₁-C₁₂)-alkyl, wherein any carbon atom is optionally replaced with one aryl group. In some embodiments, J is (C₁-C₁₂)-alkyl, wherein any carbon atom is optionally replaced with one heteroaryl group.

In some embodiments, B is a solid support. In some embodiments, B is H.

In some embodiments, X is a solid support. In some embodiments, X is H.

In some embodiments, each n is independently 1-100. In some embodiments, each n is independently 1-50. In some embodiments, each n is independently 1-10. In some embodiments, each n is independently 1-5.

In some embodiments, R is ethyl or sec-butyl; A is C₅-alkyl, B is a solid support, X is H and Y is C₁₀ alkyl. In some embodiments, R is ethyl or sec-butyl; A is n-pentyl, B is solid support, X is H and Y is n-decyl. In some embodiments, R is ethyl, A is n-pentyl, B is solid support, X is H and Y is n-decyl. In some embodiments, R is sec-butyl, A is n-pentyl, B is solid support, X is H and Y is n-decyl.

In some embodiments, R is ethyl or sec-butyl; A is C₅-alkyl, B is H, X is a solid support and Y is C₁₀ alkyl. In some embodiments, R is ethyl or sec-butyl; A is n-pentyl, B is H, X is a solid support and Y is n-decyl. In some embodiments, R is ethyl, A is n-pentyl, B is H, X is a solid support and Y is n-decyl. In some embodiments, R is sec-butyl, A is n-pentyl, B is H, X is a solid support and Y is n-decyl.

In some embodiments, R is ethyl or sec-butyl; A is —(OCH₂CH₂)ₙ—, B is a solid support, X is H and Y is C₁₀ alkyl. In some embodiments, R is ethyl or sec-butyl; A is —(OCH₂CH₂)ₙ—, B is solid support, X is H and Y is n-decyl. In some embodiments, R is ethyl, A is —(OCH₂CH₂)ₙ—, B is solid support, X is H and Y is n-decyl. In some embodiments, R is sec-butyl, A is —(OCH₂CH₂)ₙ—, B is solid support, X is H and Y is n-decyl.

In some embodiments, R is ethyl or sec-butyl; A is C₅-alkyl, B is H, X is a solid support and Y is —(OCH₂CH₂)ₙ—. In some embodiments, R is ethyl or sec-butyl; A is n-pentyl, B is H, X is a solid support and Y is —(OCH₂CH₂)ₙ—. In some embodiments, R is ethyl, A is n-pentyl, B is H, X is a solid support and Y is —(OCH₂CH₂)ₙ—. In some embodiments, R is sec-butyl, A is n-pentyl, B is H, X is a solid support and Y is —(OCH₂CH₂)ₙ—. In some embodiments, R is ethyl or sec-butyl; A is C₅-alkyl, B is H, X is H and Y is C₁₀ alkyl. In some embodiments, R is ethyl or sec-butyl; A is n-pentyl, B is H, X is H and Y is n-decyl. In some embodiments, R is ethyl, A is n-pentyl, B is H, X is H and Y is n-decyl. In some embodiments, R is sec-butyl, A is n-pentyl, B is H, X is H and Y is n-decyl.

In addition, development of novel fluorescent resonance energy transfer (FRET) reporter substrates for in vitro assays of DAGL activity are reported, along with utilization of such for assays of related lipases.

In some embodiments of the compound of formula (II), W is O or NH. In some embodiments of the compound of formula (II), W is O. In some embodiments of the compound of formula (II), W is NH.

In some embodiments of the compound of formula (II),

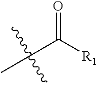

is 4-pyrenebutyryl, 10-pyrenedecanoyl, 5-doxylstearoyl, 16-doxylstearoyl, or dinitrophenyl-∈-amino-n-caproyl. In some embodiments of the compound of formula (II),

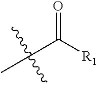

is 4-pyrenebutyryl, 10-pyrenedecanoyl, or dinitrophenyl-∈-amino-n-caproyl. In some embodiments of the compound of formula (II),

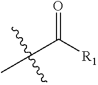

is 4-pyrenebutyryl, or dinitrophenyl-∈-amino-n-caproyl. In some embodiments of the compound of formula (II),

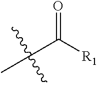

is 4-pyrenebutyryl, or 2,4-dinitrophenyl-∈-amino-n-caproyl. In some embodiments of the compound of formula (II),

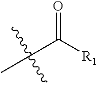

is 4-pyrenebutyryl. In some embodiments of the compound of formula (II),

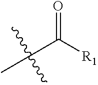

is 2,4-dinitrophenyl-∈-amino-n-caproyl.

In some embodiments of the compound of formula (II),

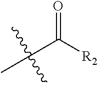

is 4-pyrenebutyryl, dinitrophenyl-∈-amino-n-caproyl, or 6-(N-(7-nityrobenz-2-oxa-1,3-diazol-4-yl)amino)hexanoyl. In some embodiments of the compound of formula (II),

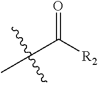

is 4-pyrenebutyryl, or dinitrophenyl-∈-amino-n-caproyl. In some embodiments of the compound of formula (II),

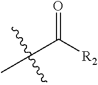

is 4-pyrenebutyryl, or 2,4-dinitrophenyl-∈-amino-n-caproyl. In some embodiments of the compound of formula (II),

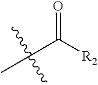

is 4-pyrenebutyryl. In some embodiments of the compound of formula (II),

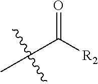

is 2,4-dinitrophenyl-∈-amino-n-caproyl.

In some embodiments of the compound of formula (II), R₃ is H or (C₁-C₁₂)-alkyl. In some embodiments of the compound of formula (II), R₃ is H or (C₁-C₆)-alkyl. In some embodiments of the compound of formula (II), R₃ is H or (C₁-C₃)-alkyl. In some embodiments of the compound of formula (II), R₃ is H or methyl. In some embodiments of the compound of formula (II), R₃ is (C₁-C₁₂)-alkyl. In some embodiments of the compound of formula (II), R₃ is (C₁-C₆)-alkyl. In some embodiments of the compound of formula (II), R₃ is (C₁-C₃)-alkyl. In some embodiments of the compound of formula (II), R₃ is methyl. In some embodiments of the compound of formula (II), R₃ is H.

In some embodiments of the compound of formula (II), W is O,

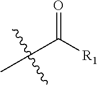

is 4-pyrenebutyryl,

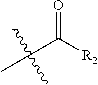

is dinitrophenyl-∈-amino-n-caproyl and R₃ is (C₁-C₃)-alkyl. In some embodiments of the compound of formula (II), W is O,

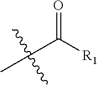

is 4-pyrenebutyryl,

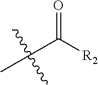

is 2,4-dinitrophenyl-∈-amino-n-caproyl and R₃ is (C₁-C₃)-alkyl. In some embodiments of the compound of formula (II), W is O,

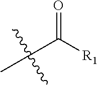

is 4-pyrenebutyryl,

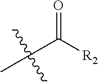

is 2,4-dinitrophenyl-∈-amino-n-caproyl, and R₃ is methyl.

In some embodiments of the compound of formula (II), W is O,

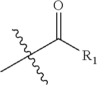

is dinitrophenyl-∈-amino-n-caproyl,

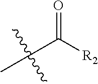

is 4-pyrenebutyryl and R₃ is (C₁-C₃)-alkyl. In some embodiments of the compound of formula (II), W is O,

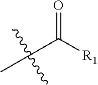

is 2,4-dinitrophenyl-∈-amino-n-caproyl,

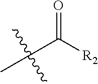

is 4-pyrenebutyryl and R₃ is (C₁-C₃)-alkyl. In some embodiments of the compound of formula (II), W is O,

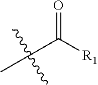

is 2,4-dinitrophenyl-∈-amino-n-caproyl,

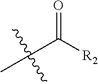

is 4-pyrenebutyryl, and R₃ is methyl.

In one aspect, the invention is directed to compositions comprising a compound of formula (I) and a pharmaceutically acceptable carrier.

In one aspect, the invention is directed to compositions comprising a compound of formula (II) and a pharmaceutically acceptable carrier.

In another aspect, the invention is directed to a method of treating a disorder in a subject comprising administration of a therapeutically effective amount of a compound of formula (I). In some embodiments, the disorder is pancreatitis or obesity. In some embodiments, the disorder is pancreatitis. In some embodiments, the disorder is obesity.

In some embodiments, a compound of formula (I) is administered. In some embodiments, a composition comprising a compound of formula (I) is administered.

Compounds of formula (I) can be incorporated into pharmaceutical compositions suitable for administration. Such compositions can comprise a compound of formula (I) and a pharmaceutically acceptable carrier. Thus, in some embodiments, the compounds of the invention are present in a pharmaceutical composition.

An ether lipid substrate having the formula (II) is added in DMSO or other organic solvent to an aqueous buffer containing protein to be assayed for lipase activity. In some embodiments, lipase activity is that of diacylglycerol lipase, lipoprotein lipase, pancreatic lipase, and related lipases. In some embodiments, lipase activity is that of diacylglycerol lipase, lipoprotein lipase, or pancreatic lipase. In some embodiments, lipase activity is that of diacylglycerol lipase. In some embodiments, lipase activity is that of lipoprotein lipase. In some embodiments, lipase activity is that of pancreatic lipase. An appropriate instrument is used with an excitation at the appropriate wavelength of light and fluorescence emission observed. The effect of the protein on the fluorescence resonance energy transfer (FRET) of the ether lipid reporter compound and its hydrolysis products assays lipase activity. This technique can be utilized in 96 well or greater formats for any appropriate instrumentation for high-throughput analysis of lipase activities. This method includes assaying the effects of added compounds as inhibitors or modulators of any lipases using the claimed reporter compounds in combination with any instrumentation.

As discussed above, the compounds of this disclosure having the formula (I), have been discovered as inhibitors of diacylglycerol lipase. This application also provides methods for inhibiting diacylglycerol lipase comprising contacting diacylglycerol lipase with at least one compound having the above formula. A reduction in the activity of diacylglycerol lipase is indicative that the diacylglycerol lipase is inhibited. The compounds of this disclosure are useful for these methods both in vivo and in vitro.

In certain embodiments, the compounds disclosed herein are attached to solid supports. Exemplary solid supports include inert substrates, inert matrices, glass slides, polymer beads, plastic tubing, glass tubing, rubber tubing, or any surface. In certain embodiments, the solid support has been “functionalized.” In some embodiments, the solid support is functionalized by application of a layer or coating of an intermediate material comprising reactive groups which permit covalent attachment of the compounds disclosed herein to the solid support.

In some embodiments, the device to treat blood will utilize a solid support such as medical grade polyvinyl chloride (a copolymer of vinyl chloride, ethylene, vinyl acetate, carbon monoxide, plasticizer, and other minor components) or other derivatizable polymer that is compatible with contact to blood. The linker molecule will be covalently attached to the polymer surface. The spacer can contain any group (-J-) or (—V—) which can be, for example, methylene (—CH₂)ₙ—), polyethylene glycol (—CH₂CH₂O)ₙ—), or any other group to control the distance of the active pharmaceutical ingredient (API) from the surface of the polymer. An example of a linker is such that a hydroxyl (HO—), sulfhydryl (HS—), or other group of the linker covalently derivatizes the polyvinyl chloride surface under basic conditions similar to known derivatizations of polyvinyl chloride (Polymer Bull. 1996, 36, 13; J Pharm Pharmacol 2009, 61, 1163; each herein incorporated by reference in its entirety) as shown in Scheme L-1, the use of thiols is generally accompanied by less dehydrochlorination of the polyvinyl chloride. The linker advantageously contains a functional group that will allow the covalent attachment of the API (drug). The lactone ring of the tetrahydrolipstatin derivative (API) shown in Scheme L-1 is stable in acidic and neutral conditions, and thermally stable below 70 degrees centigrade. This dihydrolipstatin derivative is easily obtained by the partial reduction of the corresponding alkyne (Chem. Asian J. 2011, 6, 2762 and J. Am. Chem. Soc. 2010, 132, 656; each herein incorporated by reference in its entirety). The coupling of the spacer tetrazole to the API olefin is a photoinduced (“photoclick”) reaction (ACIEE 2009, 48, 5330; Acc. Chem. Res. 2011, 44, 828; each herein incorporated by reference in its entirety) that proceeds fast, at ambient temperature, with photolysis at 302 nm, and covalently attaches the API with loss of N2. The “photoclick” attachment of non-terminal olefins of tetrahydrolipstatin and other semi-synthetic analogs obtained from fermentations is also covered. The “photoclick” coupling of a tetrazole spacer to an alkyne API is a second example of the device. The “click” coupling of an azide spacer to an alkyne API is a third example of the device. The attachment of other API (PLA2 inhibitors, protease inhibitors, or other enzyme inhibitors not limited to those listed below) via functional groups (olefin, alkyne, alcohol, thiol, halide, or other) of analog molecules with the corresponding activities for the treatment of acute pancreatitis, shock, and related diseases are equivalent forms of the device.

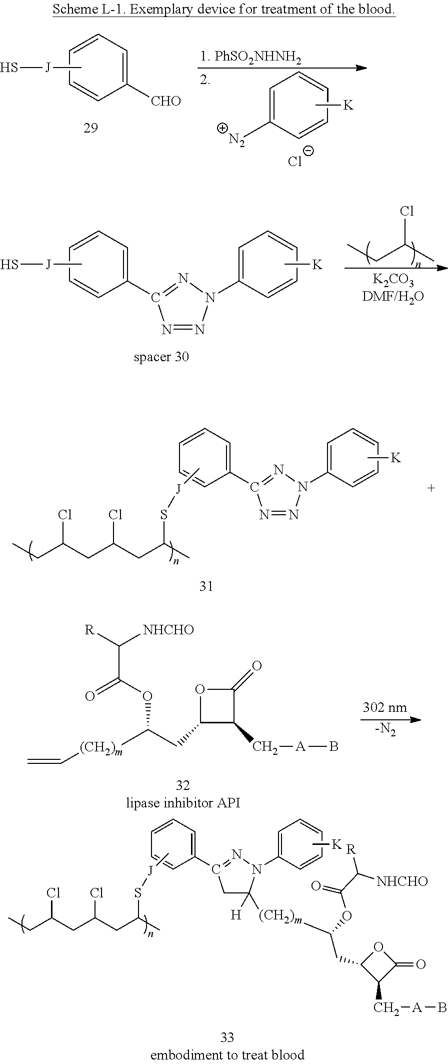

Tetrazole spacer 30. A solution of aldehyde 29 (1 eq) in ethanol was added to a solution of phenylsulfonylhydrazine (1 eq) in ethanol. After one hour, water was added and the product phenylhydrazone collected by filtration. The phenylhydrazone was dissolved in a solution of sodium hydroxide (4 eq) in ethanol, cooled to 0° C., and a solution of aryldiazonium salt (1 eq, freshly prepared from 1 eq of aniline analog and 1 eq of sodium nitrite in 1:10 concentrated aqueous HCl water) was added dropwise over 30 min. The reaction mixture was extracted with chloroform. The chloroform phase was backwashed with dilute aqueous HCl followed by water. The organic layer was dried with MgSO4, filtered, and concentrated. Purification by chromatography gave the tetrazole spacer 30.

Attached spacer to polyvinylchloride solid support 31. The polyvinyl chloride (PVC) surface was allowed to come in contact with a solution of tetrazole spacer 30 (1 eq) and potassium carbonate (1 eq) in 5:1 dimethylformamide/water. The mixture was heated to 60° C. for 6 hr. The modified PVC surface was rinsed with deionized water followed by rinsing with diethyl ether. The modified PVC was then dried with reduced pressure.

Solid-supported pancreatic lipase inhibitor device to treat blood 33. The terminal olefinic analog 32 of the corresponding alkyne (Yang, P.-Y.; Liu, K.; Zhang, C.; Chen, G. Y. J.; Shen, Y.; Ngai, M. H.; Lear, M. J.; Yao, S. Q. Chem. Asian J. 2011, 6, 2762. Yang, P.-Y.; Liu, K.; Ngai, M. H.; Lear, M. J.; Wenk, M. R.; Yao, S. Q. J. Am. Chem. Soc. 2010, 132, 656.) was prepared by hydrogenation using palladium in the presence of quinoline followed by chromatographic purification. A solution of olefin 32 in 1:1 acetonitrile/water was allowed to come in contact with modified PVC surface 31. Ultraviolet light of wavelength 302 nm was applied for two minutes to caryout the “photoclick” reaction. The device was washed with deionized water followed by rinsing with diethyl ether. The device was dried with reduced pressure.

Embodiments of the device to be delivered (orally or directly via tube (gastric, nasogastric, or related)) to the duodenum are distinct from previous reports of enzymes covalently attached to solid supports (which are primarily for the purpose of recovery and re-use of enzymes), in that, for example, the API is covalently attached to the solid support to covalently bond (or bind with high-affinity) to an enzyme for the purpose of inactivating the enzyme for medical treatment.

These embodiments are distinct from previous reports of API being adsorbed (non-covalently) to polymeric excipients for the purposes of controlled release.

A biocompatible polymer covalently attached to a linker that is covalently attached to the active pharmaceutical ingredient (API) or ingredients (APIs) will inactivate pancreatic lipase enzyme (or other pancreatic enzymes) as they are secreted into the duodenum and thus protect the small intestine from enzymatic activity resulting in damage to the intestinal wall. The solid support can be any monomethoxypolyethyleneglycol chloride, cellulose chloride (J. Org. Chem. 1958, 23, 1716; J. Polymer Sci. A 1990, 28, 2223; each herein incorporated by reference in its entirety) as shown in Scheme L-2, or other orally biocompatible non-absorbable polymer. The spacer is covalently attached to the solid support and can be an alcohol, thiol, or other compound such as described above for the blood-treatment embodiment of the device. The linker advantageously contains a functional group that will allow the covalent attachment of the API (drug) or APIs (drugs). As described above, unsaturated lipstatin analogs containing a double bond can be attached by a “photoclick” reaction as illustrated in Scheme L-2. The attachment of other lipstatin analogs and/or other API (PLA2 inhibitors, protease inhibitors, or other enzyme inhibitors not limited to those listed below) via functional groups (olefin, alkyne, alcohol, thiol, halide, or other) of analog molecules with the corresponding activities for the treatment of acute pancreatitis, shock, and related diseases are equivalent forms of the device.

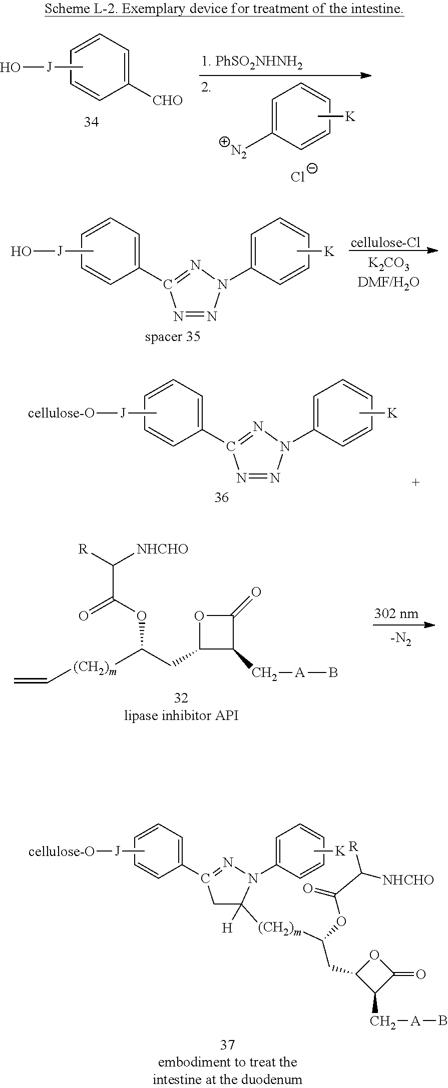

Tetrazole spacer 35. A solution of aldehyde 34 (1 eq) in ethanol was added to a solution of phenylsulfonylhydrazine (1 eq) in ethanol. After one hour, water was added and the product phenylhydrazone collected by filtration. The phenylhydrazone was dissolved in a solution of sodium hydroxide (4 eq) in ethanol, cooled to 0° C., and a solution of aryldiazonium salt (1 eq, freshly prepared from 1 eq of aniline analog and 1 eq of sodium nitrite in 1:10 concentrated aqueous HCl water) was added dropwise over 30 min. The reaction mixture was extracted with chloroform. The chloroform phase was backwashed with dilute aqueous HCl followed by water. The organic layer was dried with MgSO4, filtered and concentrated. Purification by chromatography gave the tetrazole spacer 35.

Attached spacer to cellulose solid support 36. The cellulose chloride (prepared from cotton, thionyl chloride, and pyridine according to Boehm, R. L. J. Org. Chem. 1958, 23, 1716) surface was allowed to come in contact with a solution of tetrazole spacer 35 (1 eq) and potassium carbonate (1 eq) in 5:1 dimethylformamide/water. The mixture was heated to 60° C. for 6 hr. The modified cellulose was collected by filtration, and rinsed with deionized water followed by rinsing with diethyl ether. The modified cellulose was then dried with reduced pressure.

Solid-supported pancreatic lipase inhibitor device to treat intestine 37. The terminal olefinic analog 32 of the corresponding alkyne (Yang, P.-Y.; Liu, K.; Zhang, C.; Chen, G. Y. J.; Shen, Y.; Ngai, M. H.; Lear, M. J.; Yao, S. Q. Chem. Asian J. 2011, 6, 2762. Yang, P.-Y.; Liu, K.; Ngai, M. H.; Lear, M. J.; Wenk, M. R.; Yao, S. Q. J. Am. Chem. Soc. 2010, 132, 656.) was prepared by hydrogenation using palladium in the presence of quinoline followed by chromatographic purification. A solution of olefin 32 in 1:1 acetonitrile/water was allowed to come in contact with modified cellulose surface 36. Ultraviolet light of wavelength 302 nm was applied for two minutes to caryout the “photoclick” reaction. The device was collected by filtration, and washed with deionized water followed by rinsing with diethyl ether. The device was dried with reduced pressure.

Exemplary inhibitors of PLA2 are epoxides (U.S. Pat. No. 4,788,304; herein incorporated by reference in its entirety); 3-(4-octadecyl)-benzoylacrylic acid (also OBAA, also 4-(4-octadecylphenyl)-4-oxobutenoic acid, CAS NO. 134531-42-3); 8-methoxy-6-nitrophenanthrol(3,4-d)-1,3-dioxide-5-carboxylic acid (also aristolochic acid CAS NO. 313-67-7, aristolochic acid sodium salt CAS NO. 10190-99-5); 2-[[1-oxo-3-(4-pentylphenyl)-2-propen-1-yl]amino]-benzoic acid (also ACA, also N-(4-pentylcinnamoyl)anthranilic acid, CAS NO. 99196-74-4) and analogs including the chloro analog; and 2-(p-amylcinnamoyl)amino-4-chlorobenzoic acid (also ONO-RS-082, CSA NO. 99754-06-0).

Exemplary inhibitors of PLA2/chymotrypsin are (E)-6-(bromomethylene)tetrahydro-3-(1-naphthalenyl)-2H-pyran-2-one (also bromoenollactone, also BEL, also haloenol lactone suicide substrate, also HELSS, CAS NO. 88070-98-8).

Exemplary inhibitors of PLA2/trypsin are 4-[[6-[(Aminoiminomethyl)amino]-1-oxohexyl]oxy]-benzoic acid ethyl ester salts (also ethyl 4-(6 guanidinohexanoyloxy)benzoate salts, also gabexate mesylate, CAS NO. 56974-61-9).

Exemplary inhibitors of protease are Na-tosyl-Phenylalanine-chloromethylketone (also Na-tosyl-Phe-chloromethylketone, also TPCK, CAS NO. 402-71-1); Na-tosyl-Lysine-chloromethylketone (also Na-tosyl-Lys-chloromethylketone, also TLCK, also hydrochloride salt, CAS NO. 4238-41-9); 4-(4,5-dihydro-1H-imidazol-2-ylamino)benzoic acid 6-amidinonaphthalen-2-yl ester dimethanesulfonate (also 6-[4-(2-imidazolidinylideneamino)benzoyloxy]naphthalene-2-carboxamidine dimethanesulfonate (6-carbamimidoylnaphthalen-2-yl) 4-guanidinobenzoate, also FUT-187, also TO-187, CAS NO. 103926-82-5, Related CAS: 103926-81-4 (diHCl), 103926-64-3 (free base)) (Arzneimittelforschung 1990, 40, 1352; Jap. J. Pharmacol. 1990, 52, 23; each herein incorporated by reference in its entirety); benzamidine (CAS NO. 18-39-3); and leupeptin (CAS NO. 103476-89-7).

The compounds disclosed herein may also be provided with a pharmaceutically acceptable carrier. A pharmaceutically acceptable carrier may contain inert ingredients. The pharmaceutically acceptable carriers should be non-toxic and devoid of as many other undesired reactions upon the administration to a subject as possible. Standard pharmaceutical formulation techniques can be employed, such as those described in Remington's Pharmaceutical Sciences. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents,” John Wiley and Sons, 1986).

Non-limiting examples of an effective amount of the compounds disclosed herein are provided. In a specific embodiment, the methods comprise administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds disclosed herein once every day, preferably, once every 2 days, once every 3 days, once every 4 days, once every 5 days, once every 6 days, once every 7 days, once every 8 days, once every 10 days, once every two weeks, once every three weeks, or once a month. The dosages of the compounds disclosed herein can be used in combination therapies with other drugs.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

It will recognized that one or more features of any embodiments disclosed herein may be combined and/or rearranged within the scope of the invention to produce further embodiments that are also within the scope of the invention. Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are illustrative only, since alternative methods can be utilized to obtain similar results.

### Example 1

### Synthesis of Novel Diacylglycerol Lipase Inhibitors

THL (3) has an N-formyl-L-leucyl ester and OMDM-188 (4) is the corresponding N-formyl-L-isoleucyl ester. The three other novel isoleucine diastereomers (5, 6, 7) as well as the (S)-α-aminobutyryl ester (8) were prepared via the method reported in J. Med. Chem. 2008, 51, 6970 (herein incorporated by reference in its entirety) from the corresponding benzyloxycarbonyl protected α-amino acids (13) (Scheme 1). The β-lactone analog (9) completely lacking both the N-formyl-α-aminoacyloxy and the 2-hexyl groups was prepared by treating racemic 3-hydroxypalmitic acid with N-phenyl-bis(trifluoromethanesulfonimide). The shorter chain separable trans-(10) and cis-(11) β-lactones were prepared according to the method reported in Liebigs Ann. Chem. 1996, 881; herein incorporated by reference in its entirety.

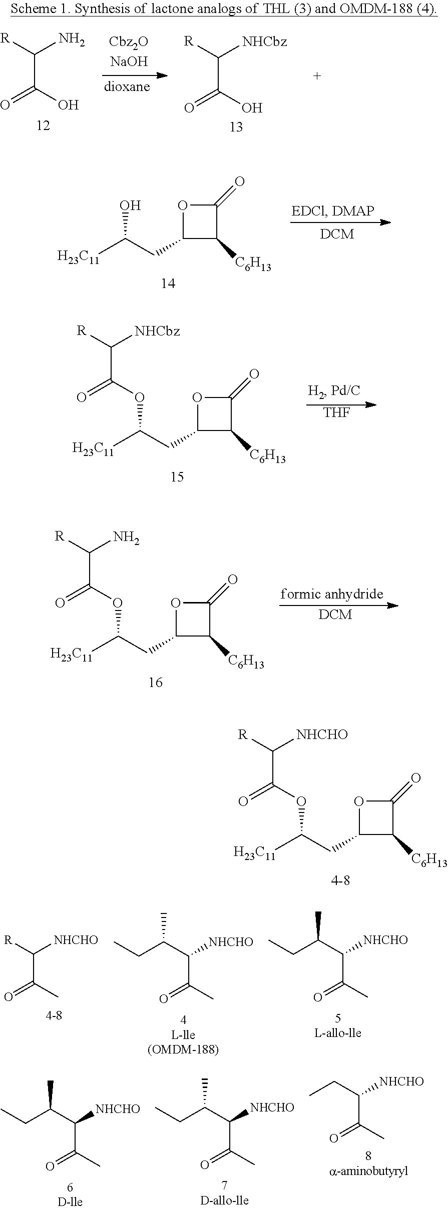

All compounds were chromatographically purified by chromatography on silica gel 60, are white solids (unless otherwise noted), and had the expected IR absorbance peaks. Observed rotations were too low to be accurately determined at the 0.1 g 100 mL CH₂Cl₂ concentrations used. All proton NMR spectra were in CDCl₃ at 500 MHz. The three other novel isoleucine diastereomers (5, 6, 7) as well as the novel (S)-α-aminobutyryl ester 8 were prepared via the reported method using alcohol 14 (Ortar, G.; Bisogno, T.; Ligresti, A.; Morera, E.; Nalli, M.; Di Marzo, V. J. Med. Chem. 2008, 51, 6970) and the corresponding benzyloxycarbonyl protected α-amino acids 13. No previous publication of THL (3) to our knowledge has noted the small percentage of trans-N-formate present that does not interconvert with tile cis-N-formate isomer at temperatures below the decomposition temperatures for THL and the related β-lactones. The cis and trans formates were not chromatographically isolated, nor was the D-allo-isoleucyl analog (7) separated from the D-isoleucyl analog (6).

(3) THL mp 41-42° C.; ¹H NMR δ 0.88 (t, J=6.4 Hz, 3H), 0.89 (t, J=6.4 Hz, 3H), 0.97 (d, J=5.9 Hz, 3H), 0.98 (d, J=5.9 Hz, 3H), 1.22-1.50 (m, 27H), 1.50-1.87 (m, 6H), 2.00 (ddd, J=15.1, 4.9, 4.9 Hz, 1H), 2.17 (ddd, J=15.2, 7.8, 7.8 Hz, 1H), 3.22 (ddd, J=7.3, 7.3, 3.9 Hz, 1H), 4.29 (ddd, J=7.8, 4.9, 3.9 Hz, 1H), 4.70 (ddd, J=9.0, 8.6, 4.9 Hz, 1H), 5.00-5.06 (m, 1H), 5.89 (d, J=8.4 Hz, 1H), 8.22 (s, 1H), trans isomer 0.95 (d, J=7.8 Hz, 0.15H), 4.10 (ddd, J=9.8, 9.7, 4.4 Hz, 0.05H), 5.05-5.11 (m, 0.05H), 5.72 (dd, J=11.7, 10.3 Hz, 0.05H), 8.06 (d, J=11.7 Hz, 0.05H).

L-isoleucyl analog 4 (OMDM188) Formic anhydride (79.3 mg, 1.07 mmol) was added to (αS,βS)-amine 16 (50.1 mg, 0.107 mmol) in dry DCM. The solution was magnetically stirred at kept at reduced temperatures between −5° C. and −10° C. for two hours. The reaction mixture was monitored by TLC (1:9 ethyl acetate/CH₂Cl₂: product Rf 0.30). After consumption of starting material, the reaction mixture was subjected to an aqueous workup and extraction with DCM. The organic layer was dried with MgSO₄ and concentrated under reduced pressure. Purification by column chromatography (1:9 ethyl acetate/CH₂Cl₂) gave a 7.2% yield. mp 59-60° C.; lit (Ortar et al. JMC 2008, 51, 6970) mp 57-59° C.; ¹H NMR δ 0.88 (t, J=7.3 Hz, 3H), 0.89 (t, J=7.3 Hz, 3H), 0.96 (t, J=7.8 Hz, 3H), 0.97 (d, J=7.8 Hz, 3H), 1.10-1.60 (m, 28H), 1.58-1.84 (m, 4H), 1.85-2.00 (m, 1H), 2.02 (ddd, J=15.0, 4.5, 4.5 Hz, 1H), 2.19 (ddd, J=14.9, 7.7, 7.7 Hz, 1H), 3.24 (ddd, J=7.4, 7.4, 4.2 Hz, 1H), 4.29 (ddd, J=7.7, 4.5, 4.2 Hz, 1H), 4.68 (dd, J=8.8, 4.9 Hz, 1H), 5.00-5.07 (m, 1H), 6.05 (d, J=8.8 Hz, 1H), 8.26 (s, 1H). trans isomer 0.99 (d, J=7.8 Hz, 0.15H), 3.97 (dd, J=10.2, 4.9 Hz, 0.05H), 5.06-5.11 (m, 0.05H), 5.87 (dd, J=11.7, 10.3 Hz, 0.05H), 8.03 (d, J=11.7 Hz, 0.05H).

L-allo-isoleucyl analog 5. Formic anhydride (148.8 mg, 2.0 mmol) was added to (αS,βR)-amine 16 (47.0 mg, 0.10 mmol) in dry DCM. The solution was magnetically stirred at kept at reduced temperatures between −5° C. and −10° C. for two hours. The reaction mixture was monitored by TLC (1:9 ethyl acetate/CH₂Cl₂). Purification by column chromatography (1:9 ethyl acetate/CH₂Cl₂) gave a 55% yield; mp 49-50° C.; ¹H NMR δ 0.87 (t, J=7.3 Hz, 3H), 0.88 (t, J=7.3 Hz, 3H), 0.96 (t, J=7.6 Hz, 3H), 0.97 (d, J=7.6 Hz, 3H), 1.14-1.52 (m, 28H), 1.54-1.88 (m, 4H), 1.92-2.00 (m, 1H), 2.02 (ddd, J=15.1, 5.4, 5.4 Hz, 1H), 2.19 (ddd, J=15.1, 7.7, 7.5 Hz, 1H), 3.23 (ddd, J=7.6, 7.6, 3.9 Hz, 1H), 4.29 (ddd, J=7.6, 5.4, 3.9 Hz, 1H), 4.77 (dd, J=9.0, 3.7 Hz, 1H), 4.96-5.05 (m, 1H), 6.10 (d, J=8.8 Hz, 1H), 8.26 (s, 1H). trans isomer 4.07 (dd, J=10.2, 3.9 Hz, 0.05H), 5.04-5.11 (m, 0.05H), 6.01 (dd, J=11.7, 10.3 Hz, 0.05H), 8.00 (d, J=11.7 Hz, 0.05H).

D-isoleucyl analog 6. Formic anhydride (85.6 mg, 1.15 mmol) was added to (αR,βR)-amine 16 (15.46 mg, 0.033 mmol) in dry DCM. The solution was magnetically stirred at kept at reduced temperatures between −5° C. and −10° C. for two hours. The reaction mixture was monitored by TLC (1:9 ethyl acetate/CH₂Cl₂: product Rf 0.30). After consumption of starting material, the reaction mixture was concentrated. Purification by column chromatography (1:9 ethyl acetate/CH₂Cl₂) gave a 16% yield of a clear and colorless liquid; ¹H NMR δ 0.89 (t, J=6.6 Hz, 6H), 0.95 (t, J=7.3 Hz, 3H), 0.97 (d, J=7.3 Hz, 3H), 1.14-1.52 (m, 28H), 1.54-1.88 (m, 4H), 1.92-2.00 (m, 1H), 2.03 (ddd, J=14.9, 4.5, 4.5 Hz, 1H), 2.19 (ddd, J=14.9, 7.9, 6.5 Hz, 1H), 3.24 (ddd, J=7.4, 7.4, 4.2 Hz, 1H), 4.34 (ddd, J=7.9, 4.5, 4.2 Hz, 1H), 4.66 (dd, J=8.6, 4.6 Hz, 1H), 5.01-5.08 (m, 1H), 6.03 (d, J=8.8 Hz, 1H), 8.25 (s, 1H). trans isomer 1.00 (t, J=7.3 Hz, 0.15H), 2.05 (ddd, J=14.9, 4.5, 4.5 Hz, 0.05H), 3.24 (ddd, J=7.4, 7.4, 4.2 Hz, 0.05H), 3.99 (dd, J=10.3, 4.3 Hz, 0.05H), 4.26-4.32 (m, 0.05H), 5.09-5.17 (m, 0.05H), 5.98 (dd, J=11.7, 10.3 Hz, 0.05H), 8.02 (d, J=11.7 Hz, 0.05H). D-isoleucine has second 5% impurity of D-alloisoleucine from starting amino acid 4.77 (dd, J=9.0, 3.7 Hz, 0.05H), 5.98 (d, J=8.8 Hz, 0.05H), 8.27 (s, 0.05H).

D-allo-isoleucyl analog 7. Formic anhydride (142.45 mg, 1.92 mmol) was added to (αR,βS)-amine 16 (45.0 mg, 0.096 mmol) in dry DCM. The solution was magnetically stirred at kept at reduced temperatures between −5° C. and −10° C. for 45 minutes. At this time diisopropylethylamine (12.4 mg, 0.016 mL) was added before being stirred at room temperature for 1 hour. The reaction mixture was monitored by TLC (1:9 ethyl acetate/CH₂Cl₂: product Rf 0.30). After consumption of starting material, the reaction mixture was concentrated. Purification was by column chromatography (1:9 ethyl acetate/CH₂Cl₂) gave a clear and colorless liquid; ¹H NMR δ 0.85-0.90 (m, 9H), 0.97 (t, J=7.3 Hz, 3H), 1.16-1.51 (m, 28H), 1.52-1.87 (m, 4H), 1.91-2.01 (m, 1H), 2.02 (ddd, J=15.0, 5.0, 4.9 Hz, 1H), 2.19 (ddd, J=15.0, 7.7, 7.1 Hz, 1H), 3.23 (ddd, J=7.4, 7.3, 3.9 Hz, 1H), 4.33 (ddd, J=7.7, 5.0, 3.9 Hz, 1H), 4.77 (dd, J=9.0, 3.7 Hz, 1H), 5.00-5.08 (m, 1H), 5.96 (d, J=8.8 Hz, 1H), 8.27 (s, 1H). trans isomer 0.96 (t, J=7.3 Hz, 0.15H), 2.13 (m, 0.05H), 3.17-3.22 (m, 0.05H), 4.08 (dd, J=10.0, 3.7 Hz, 0.05H), 4.26-4.32 (m, 0.05H), 5.09-5.17 (m, 0.05H), 5.87 (dd, J=11.7, 10.0 Hz, 0.05; H), 8.00 (d, J=11.7 Hz, 0.05H).

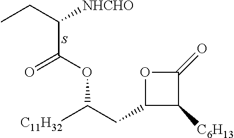

(N-formylaminobutyric acid)

α-Aminobutyryl analog 8. Formic anhydride (95.5 mg, 1.29 mmol) was added to (αS)-amine 16 (28.5 mg, 0.068 mmol) in dry DCM. The solution was magnetically stirred at kept at reduced temperatures between −5° C. and −10° C. for 45 minutes. At this time diisopropylethylamine (8.37 mg, 0.011 mL) was added before being stirred at room temperature for 1 hour. The reaction mixture was monitored by TLC (1:9 ethyl acetate/CH₂Cl₂: product Rf 0.30). After consumption of starting material, the reaction mixture was concentrated. Purification by column chromatography (1:9 ethyl acetate/CH₂Cl₂) gave a 27% yield: mp 43-44° C.; ¹H NMR δ 0.88 (t, J=6.7 Hz, 3H), 0.89 (t, J=6.7 Hz, 3H), 0.96 (t, J=7.6 Hz, 3H), 1.20-1.54 (m, 26H), 1.54-1.90 (m, 5H), 1.92-2.01 (m, 1H), 2.02 (ddd, J=15.1, 4.4, 4.4 Hz, 1H), 2.17 (ddd, J=15.1, 7.9, 7.8 Hz, 1H), 3.23 (ddd, J=7.4, 7.4, 4.2 Hz, 1H), 4.30 (ddd, J=8.0, 4.5, 4.4 Hz, 1H), 4.64 (ddd, J=7.3, 7.3, 7.3 Hz, 1H), 5.02-5.09 (m, 1H), 6.10 (d, J=7.3 Hz, 1H), 8.25 (s, 1H). isomer 1.00 (t, J=7.6 Hz, 0.15H), 4.03 (ddd, J=9.8, 7.8, 5.4 Hz, 0.05H), 5.07-5.13 (m, 0.05H), 5.87 (dd, J=11.7, 9.8 Hz, 0.05H), 8.08 (d, J=11.7 Hz, 0.05H).

4-Tridecyloxetan-2-one (9). A stirred solution of DL-β-hydroxypalmitic acid (20.0 mg, 0.0734 mmol) and TEA (14.8 mg, 0.020 mL) in dry DCM was treated with N-phenyl-bis(trifluoromethanesulfonimide (39.34 mg, 0.11 mmol) at 0° C. The resulting mixture was stirred at room temperature overnight and monitored by TLC (1:9 ethyl acetate/hexane: product Rf 4.75). Upon completion of the reaction, the mixture was concentrated and purified by column chromatography (1:9 ethyl acetate/hexane): mp 39-40° C.; ¹H NMR δ 0.88 (t, J=6.8 Hz, 3H), 1.21-1.51 (m, 22H), 1.69-1.79 (m, 1H), 1.82-1.92 (m, 1H), 3.06 (dd, J=16.4, 4.2 Hz, 1H), 3.50 (dd, J=16.1, 5.9 Hz, 1H), 4.50 (ddd, J=11.5, 6.0, 6.0 Hz, 1H).

cis-3-Hexyl-4-heptyloxetan-2-one (10) and trans-3-hexyl-4-heptyloxetan-2-one (11). Benzene sulfonyl chloride (13.6 mmol, 1.742 ml) was added drop-wise to a magnetically stirring solution of β-hydroxy acid (6.8 mmol, 1.85 g, from the condensation of octanoic acid dianion with octanal) in 28 ml of dry pyridine at 0° C. The solution was subsequently shaken, sealed and stored in the refrigerator overnight. Workup included pouring the reaction mixture over 3 volumes of crushed ice before extraction with several volumes of Et₂O. The combined ether layers were washed with saturated NaHCO₃ and water. The organic layer was dried with MgSO₄, filtered and concentrated under reduced pressure. Chromatographic separation by column chromatography (1:1 CH₂Cl₂/hexane: product Rf 0.50) gave the two products as a clear and colorless liquids: (10) trans-13-Lactone (racemic, MRJ18) clear and colorless liquid; ¹H NMR δ 0.89 (t, J=6.8 Hz, 6H), 1.20-1.50 (m, 18H), 1.66-1.77 (m, 2H), 1.77-1.95 (m, 2H), 3.16 (ddd, J=8.7, 6.4, 4.1 Hz, 1H), 4.21 (ddd, J=6.7, 6.7, 4.1 Hz, 1H).

(11) cis-β-Lactone (racemic, MRJ17) clear and colorless liquid; ¹H NMR δ 0.89 (t, J=6.8 Hz, 6H), 1.20-1.47 (m, 16H), 1.45-1.58 (m, 2H), 1.59-1.71 (m, 2H), 1.71-1.85 (m, 2H), 3.60 (ddd, J=8.9, 6.8, 6.8 Hz, 1H), 4.54 (ddd, J=9.7, 6.1, 4.2 Hz, 1H).

### Example 2

### Synthesis of Ether Lipids

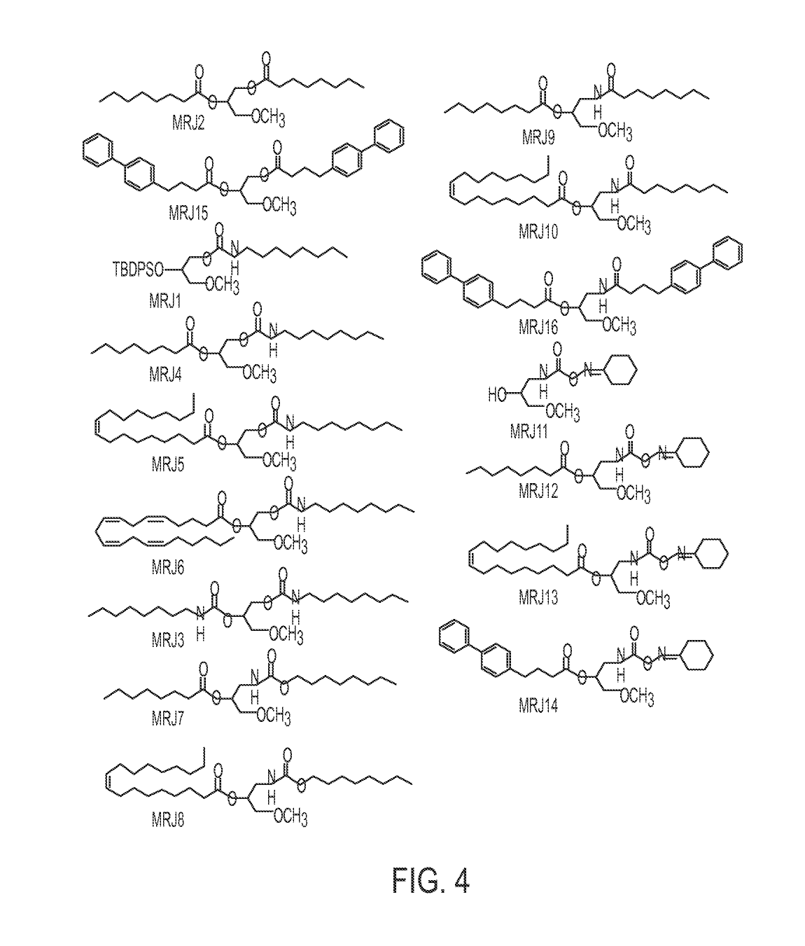

Ether lipid analogs of 0-3841 (2) were synthesized including analogs that had the reactive carbamate group of RHC80267 (1). The 3-O-methyl glycerol derivatives MRJ1 to MRJ16 (FIG. 4) were synthesized utilizing the tritylation, silyl ether protection, and detritylation conditions as shown in Schemes 1-1, 1-2 and 1-3 (See, J. Am. Chem. Soc. 2008, 130, 2722 and J. Org. Chem. 2008, 73, 9657; each herein incorporated by reference in its entirety). These compounds included analogs having ester, amide, carbamate, and cyclohexyloximinocarbamate functional groups in the sn-1 position.

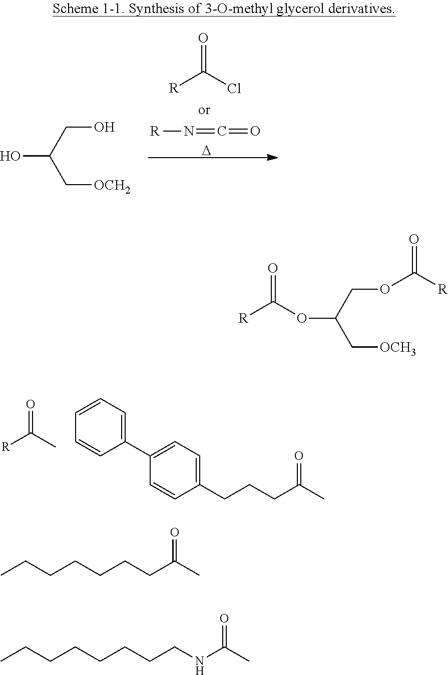

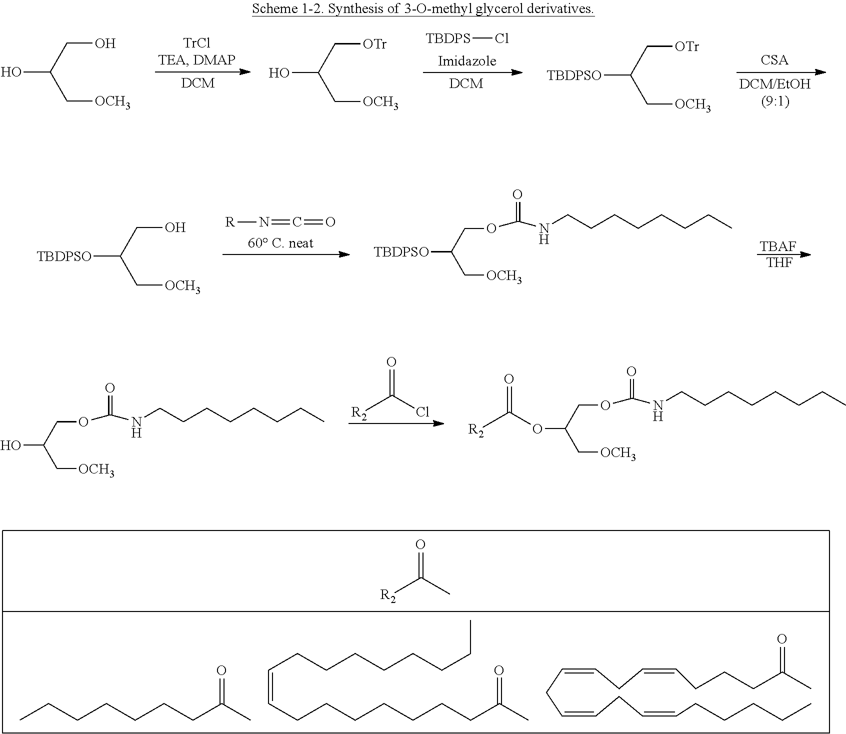

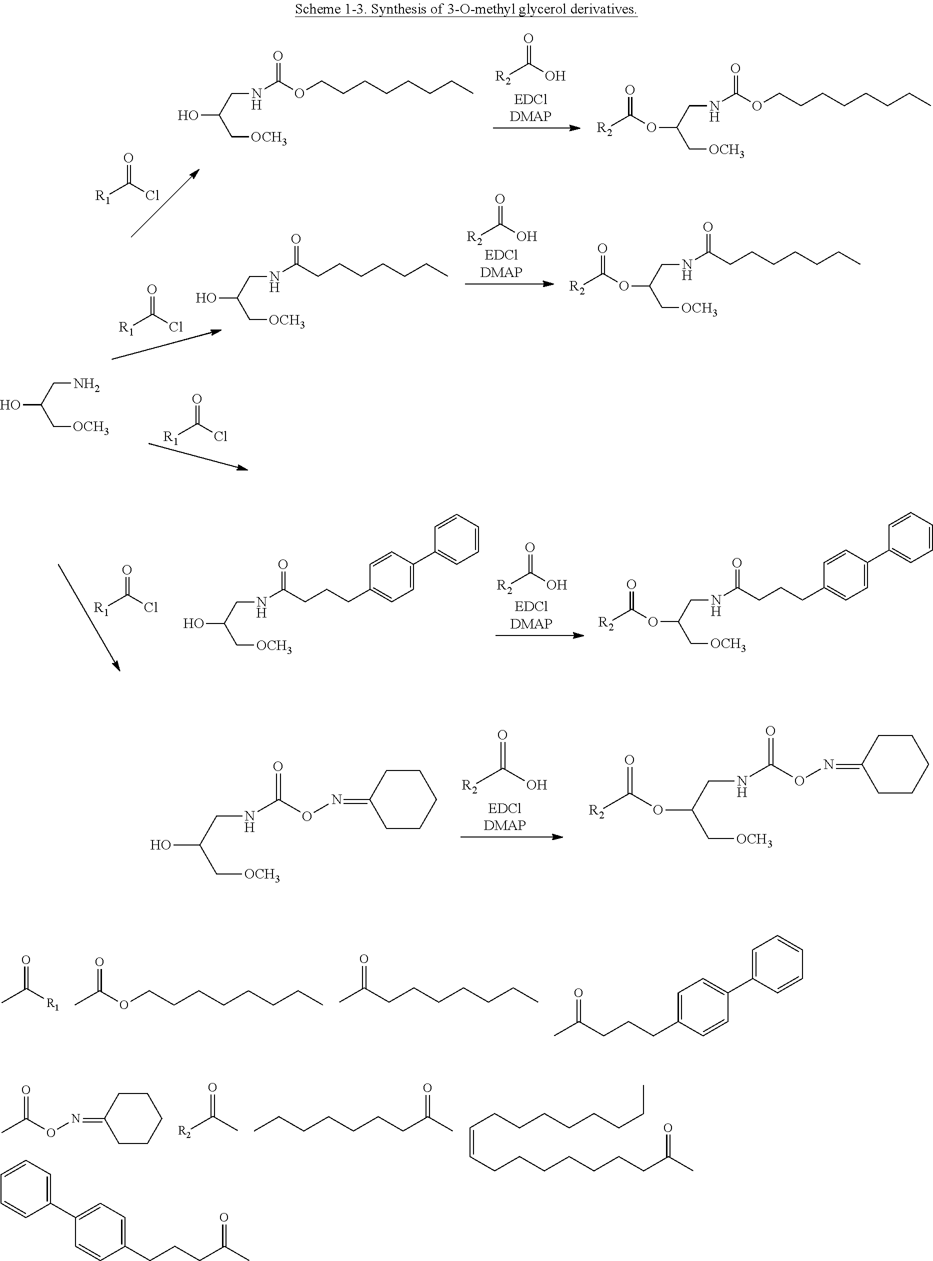

These compounds were not inhibitors of hDAGLα or mDAGLα at 10 μM. These compounds each had a Ki above 1 μM in competition binding assays for CB1 (rat brain preparation) and for CB2 (mouse or human receptor expressed in HEK293). They also did not inhibit rFAAH or hMAGL.

### Example 3

### Inhibition of Diacylglycerol Lipase

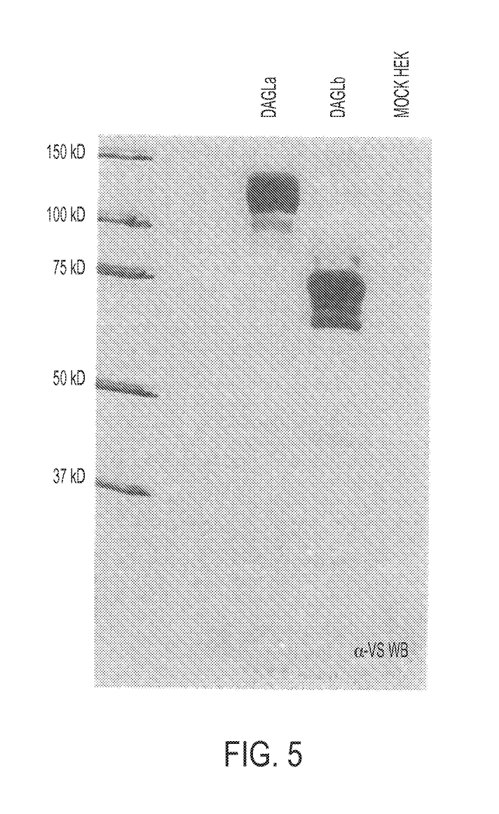

Compounds were assayed for the inhibition of diacylglycerol lipase (DAGL) activity using either cell lysates or membrane fractions that were prepared according to the previously reported method (See, J. Cell Biol. 2003, 163, 463; Eur. J. Med. Chem. 2008, 43, 62; each herein incorporated by reference in its entirety). The Cravatt group at Scripps provided hDAGLα, mDAGLα, and mDAGLβ from overexpression by transient infection of HEK293T cultures in addition to cell lysate of the empty vector HEK293T control (FIG. 5). Some experiments used a second commercially prepared plasmid to provide additional human α-isoform overexpressed in HEK293T using the same methodology. The lipase activity of hDAGLα expressed in the human cell line was sufficient for assay of newly synthesized inhibitors. At least 100 μg of total protein from crude cell lysates or at least 10 μg of total protein from membrane preparation was required per well. The proteins were utilized such that the substrate hydrolysis would proceed to the extent of about 5% in 20 min. The more readily expressed mDAGLβ isoform (that has a 79% homology with the human isoform—J. Cell Biol. 2003, 163, 463; herein incorporated by reference in its entirety) or mDAGLα isoform (that has a 97% homology with the human isoform—J. Cell Biol. 2003, 163, 463; herein incorporated by reference in its entirety) were also used to confirm inhibition of DAGL activities. The specific activities of the hDAGLα were in the range of 0.003 to 0.01 nmol/mg-min for the cell lysates (specific activity was 0.06 in the presence of 0.05% Triton X-100) and up to 0.1 nmol/min-mg for membrane (10,000×g fraction) preparations. DAGL activities of protein from empty plasmid transfections were 0.003 to 0.01 mmol/min-mg. The TLC analyses with mDAGLβ used 20 μg of protein from cell lysate with a specific activity above 0.1 nmol/min-mg. The DAGL analyses with mDAGLα used 8.8 μg of protein from a membrane preparation with a specific activity above 0.3 nmol/min-mg. The specific activity of the lipoprotein lipase positive control under the assay conditions was >400 nmol/mg-min.

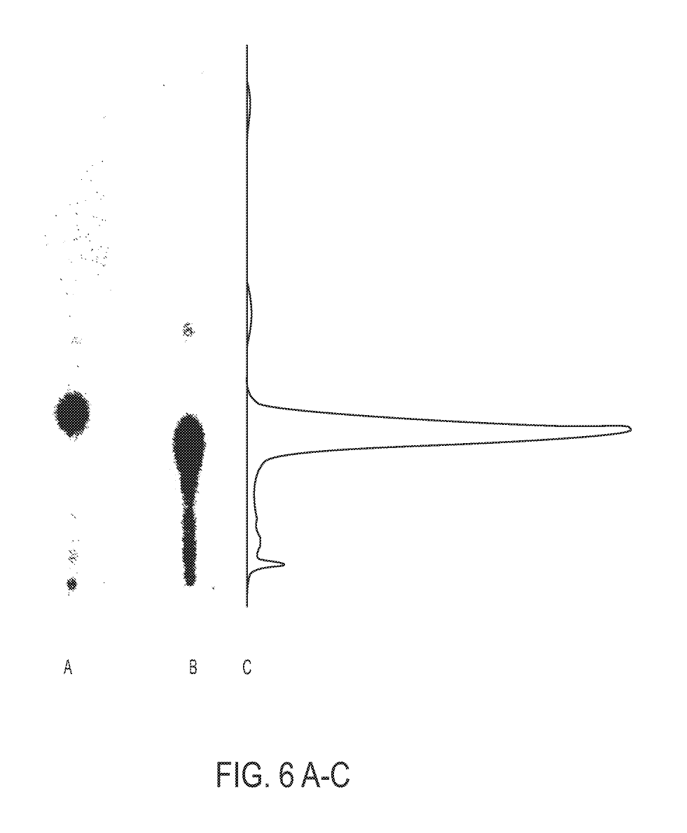

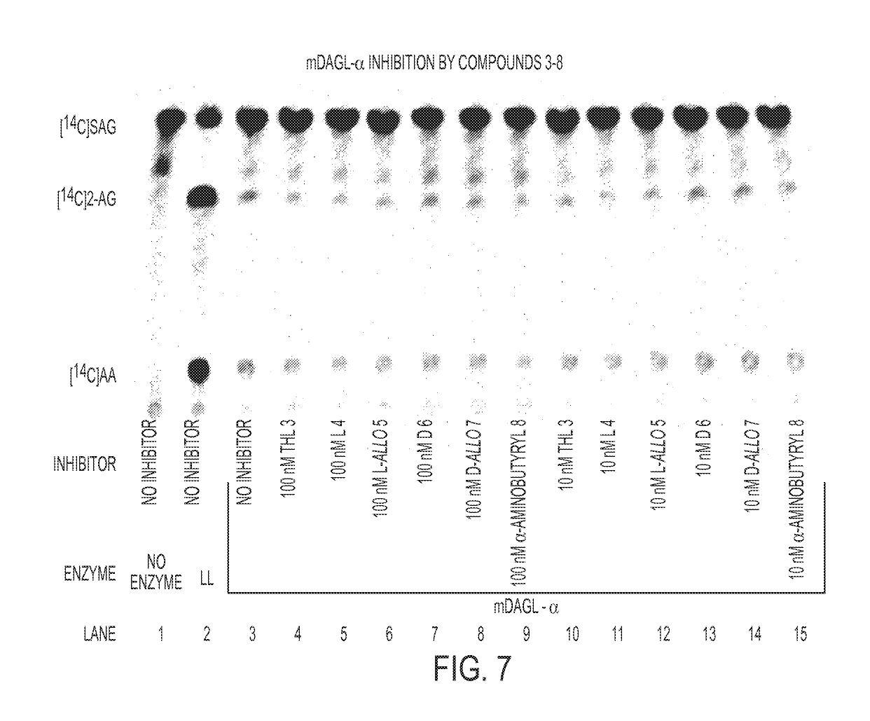

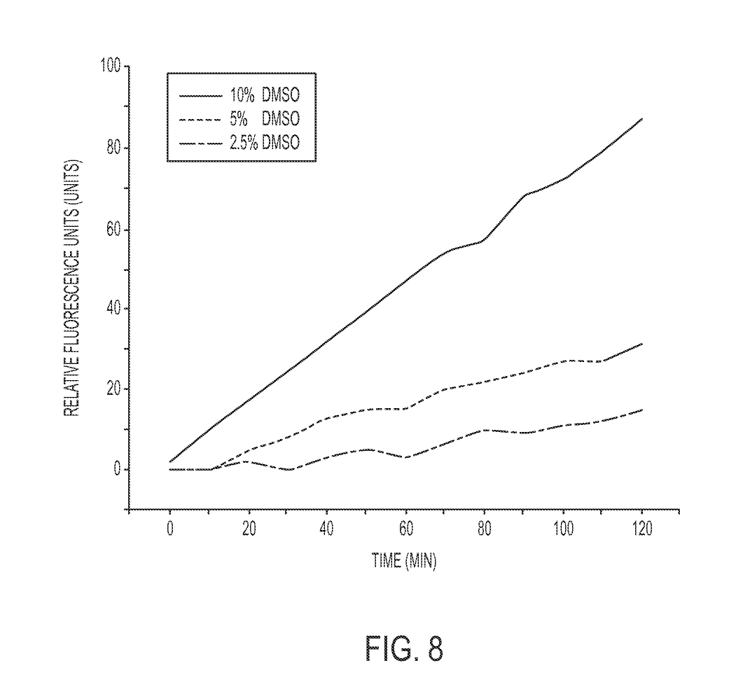

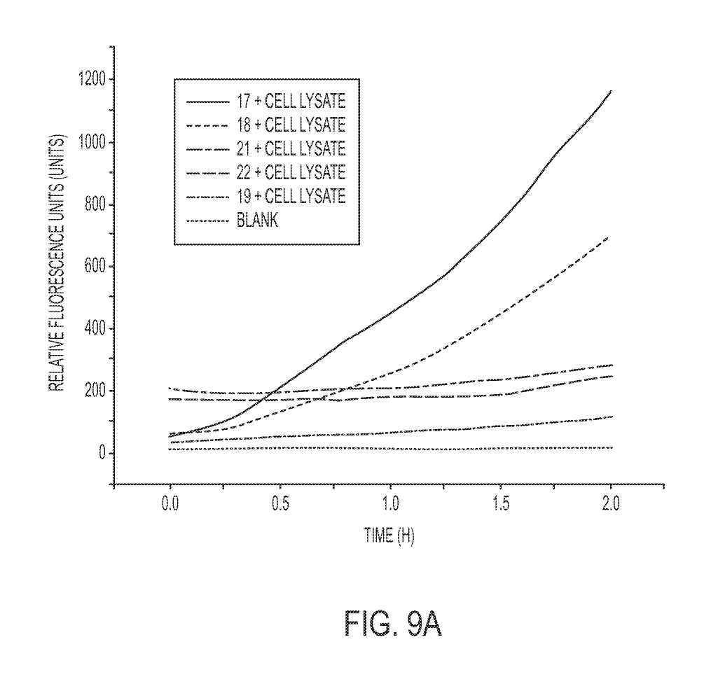

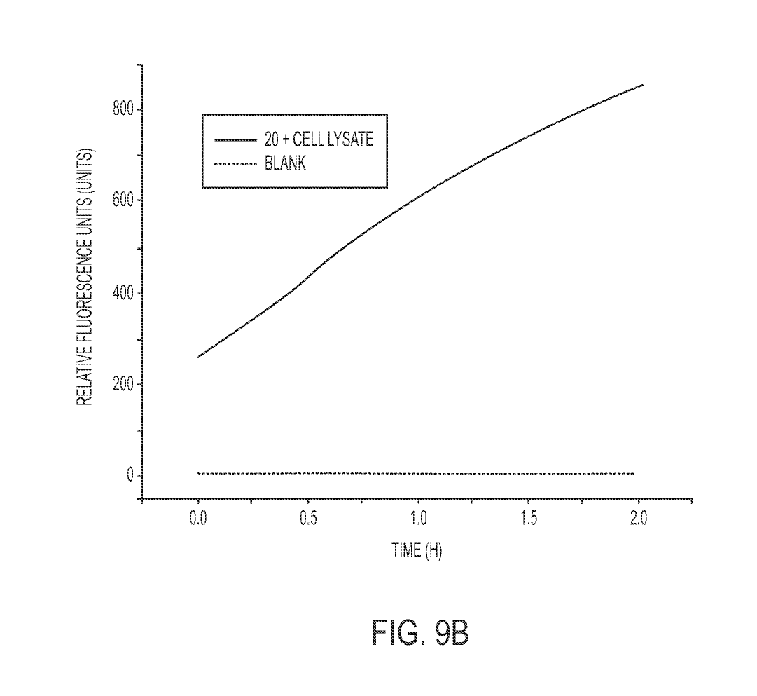

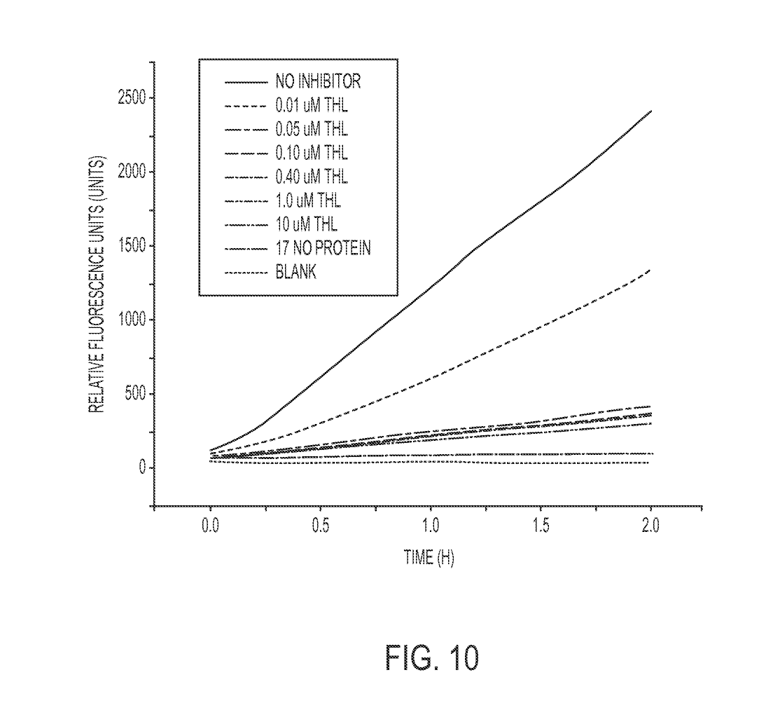

It was of importance for biological relevance and inhibitor evaluation that assays of DAGL activities utilize pure endogenous substrate such as radiolabeled [1″-¹⁴C] 1-stearoyl-2-arachidonoyl-sn-glycerol ([¹⁴C]SAG) (Methods Enzymol. 1982, 86, 11; J. Label. Compd. Radiopharm. 2009, 52, 324; each herein incorporated by reference in its entirety). Any radiolabeled 1-stearoyl-3-arachidonoyl-sn-glycerol from rearrangement of [¹⁴C]SAG to labeled 1,3-diglyceride that is present in the substrate would be readily hydrolyzed by other enzymes in the relatively crude enzyme preparations and the result misinterpreted to be due to DAGL activity. The 1,2-diglyceride substrate [¹⁴C]SAG that was used contained less than 0.5% of the 1,3-diglyceride isomer (FIG. 6A-C).

Although the [¹⁴C]SAG substrate has previously been used for TLC-based assays of DAGL activity, details of these TLC assays were not fully described (J. Cell Biol. 2003, 163, 463; Biochim. Biophys. Acta 2006, 1761, 205; Toxicol. Appl. Pharmacol. 2001, 173, 48; each herein incorporated by reference in its entirety). Also, the apparent IC₅₀ of THL (3) for hDAGLα has been reported to range from 60-1000 nM, postulated to be due to the effect of DAGL protein concentration (See, J. Cell Biol. 2003, 163, 463; Biochim. Biophys. Acta 2006, 1761, 205; J. Physiol. 2006, 577, 263; each herein incorporated by reference in its entirety). Therefore, a standardized assay was developed that contained 10% DMSO.

General.

THL (3), JZL184, URB597, and other inhibitors used were commercially available and used as freshly prepared DMSO solutions. All arachidonates were maintained under argon or in argon-degassed solvents. Glass-backed silica gel 60 TLC plates were used, and long delays between spotting and elution were avoided. All solvent ratios are by volume. All data are reported as the mean of triplicate experiments except n=1 or 2 for radio-TLC assays and FRET screenings with DAGLs, lipoprotein lipase, and pancreatic lipase. Increased enzymatic activity (rather than inhibition) is indicated by <0% inhibition, and is likely due to detergent or other effects at higher compound (>10 nM) assay concentrations as has previously been observed in DAGL assays (Nat. Chem. Biol. 2009, 5, 37; herein incorporated by reference in its entirety). All IC₅₀ data discussed and reported from the literature are actually apparent IC₅₀ as all inhibitors in this investigation undergo covalent reactions with the hydrolytic enzymes.

Radio-TLC Assay of DAGL Inhibition.

All cells, lysates (fresh or stored), and membrane preparations were probe sonicated for 5 periods of 3 s with ice bath cooling immediately prior to use. To the protein (100 μg) suspensions containing DAGL in 90 μL of buffer (50 mM Tris, pH 7.4, 10 mM CaCl₂) in screwtop eppendorfs (with O-rings) was added 5 μL of pure DMSO (for the control, 0% inhibition) or the inhibitors in 5 μL of DMSO to be assayed at their appropriate concentrations. As a positive control, 5 μL of a stable solution of lipoprotein lipase (0.23 ng, Sigma, from Pseudomonas sp. (Toxicol. Appl. Pharmacol. 2001, 173, 48; herein incorporated by reference in its entirety), 0.2% n-heptyl-β-D-thioglucopyranoside, 10 mM CaCl₂, 100 mM NaCl, 50 mM Tris, pH 7.4) was always used. Each vial was hand mixed briefly then incubated for 15 min in a sand bath at 37° C. Then, [¹⁴C]SAG substrate (304,000 dpm, specific activity 55 Ci/mol) in 5 μL of DMSO was added by microcap to all vials. An extra 5 μL sample of [¹⁴C]SAG in DMSO was always checked by scintillation counting to evaluate substrate concentration. After a brief hand mixing and 20 min incubation at 37° C., the reaction was terminated by adding 200 μL of 2:1 CHCl₃/MeOH and vortexing for 1 min. Centrifugation for 2 min at 10,000×g gave a 150 μL bottom phase that was predominantly chloroform, a small protein interphase, and an upper phase that was predominantly water. The upper phase and protein interface contained negligible (twice background) radioactive material by scintillation counting. Using a 200 μL pipette tip, approximately 100 μL of each bottom phase was transferred to new eppendorf vials. Then, 5 μL samples of the bottom phases (approximately 10,000 dpm) were spotted for TLC, and 5 μL samples were also checked by scintillation counting. The silica gel 60 TLC plates were eluted with chloroform/methanol/aqueous ammonium hydroxide. Though literature reports range from 85:15:0.1 to 85:15:1 (J. Cell Biol. 2003, 163, 463; Biochim. Biophys. Acta 2006, 1761, 205; ChemMedChem 2009, 4, 946; J. Med. Chem. 2008, 51, 6970; each herein incorporated by reference in its entirety) an optimized ratio of 86:14:0.75 elutes substrate [¹⁴C]SAG (Rf 0.88), [¹⁴C]2-AG (Rf 0.59), and [¹⁴C]arachidonic acid (Rf 0.11). In addition to the characteristic decompositions under the basic conditions of radiolabeled arachidonic acid and diglyceride substrate [¹⁴C]2-AG, there generally appeared to be more degradation of radiolabeled substrate if it did not contain carrier lipids from cell extraction. The air dried TLC plates were apposed to Perkin Elmer multisensitive screens for 12 h. Raw data as gross digital light units (DLU) were obtained from the Perkin Elmer Cyclone phosphorimaging system for quantitative analysis (OptiQuant software version 5.0) (Electrophoresis 1990, 11, 355; Biotechniques 1999, 26, 432; each herein incorporated by reference in its entirety). Percent inhibition was calculated following background subtraction. The protein specific activities were obtained from the controls. Other standard compounds from prior literature that were used, JZL 184 (Nat. Chem. Biol. 2009, 5, 37; herein incorporated by reference in its entirety), PMSF (J. Cell Biol. 2003, 163, 463; herein incorporated by reference in its entirety), and RHC80267 (J. Cell Biol. 2003, 163, 463; J. Physiol. 2006, 577, 263; J Biochem. 1999, 125, 1077; each herein incorporated by reference in its entirety) have been reported to be poor inhibitors of DAGL activity.

Higher DMSO concentrations did not increase rates of substrate conversion to fluorescent products in any of the in vitro assays. Non-denaturing detergents n-heptyl-β-D-thioglucopyranoside and Triton X-100 at concentrations up to 0.2% were also used in some experiments (see Tables 1 and 3). Detergent use dramatically increases apparent DAGL specific activities and also decreases the apparent IC₅₀ for DAGL inhibitory compounds. The DAGL enzyme suspensions had a 15 minute preincubation period of covalent quasi-irreversible inactivation by THL (3) or other potential inhibitors. The substrate [¹⁴C]SAG was then added (20 μM final concentration) and residual DAGL activity was quantified after 20 min by quenching the reaction with 2:1 chloroform/methanol and vortexing to denature the protein and move all lipids out of the aqueous phase. Very little rearrangement of [¹⁴C]SAG (1,2-diglyceride) to 1,3-diglyceride occurred under the reaction and workup conditions as assessed by TLC using boric acid treated silica gel plates.

TABLE 1
Assays of the inhibition of rFAAH, hMAGL, and hDAGLα
(radio-TLC assay with [¹⁴C]SAG substrate) enzyme activities.

| | | | | hDAGLα |
|---|---|---|---|---|
| | | | | inhibition |
| | | | | at 10 μM |
| | Detail of | rFAAH | hMAGL | TLC |
| | N-formyl- | inhibition | inhibition | detergent free or |
| Compound | α-amino | at 10 μM | at 10 μM | (with Triton X- |
| Number | ester | (%) | (%) | 100) (%) |
| 3 (THL) | L-leucyl | 6 | 47 | 100, (100^{A}) |
| 4 (OMDM- | L-isoleucyl | 7 | 16 | (98^{A}) |
| 188) | | | | |
| 5 | L-allo-isoleucyl | 3 | 1 | (95^{A}) |
| 6 | D-isoleucyl^{C} | 28 | 39 | (78^{A}) |
| 7 | D-allo- | 0 | 42 | (86^{A}) |
| | isoleucyl | | | |
| 8 | (S)-α- | 12 | 45 | (99^{A}) |
| | aminobutyryl | | | |
| 9 | none | 10 | 4 | (25^{A}, 45^{B}) |
| trans-10 | none | 79 | 56 | (37^{B}) |
| cis-11 | none | 94^{D} | 100^{D} | (25^{B}) |
| JZL184 | NA | 97^{E} | 100^{F} | (37^{B}) |
| URB597 | NA | 100^{E} | 18 | ND |
| n- | NA | 100^{E} | 82 | ND |
| C₁₆H₃₃SO₂F | | | | |
| PMSF | NA | 100^{E} | 5 | 19 |
| RHC80267 | NA | 95^{E} | 22 | 30 |
| (1) | | | | |
| SD41 | NA | 10 | 13 | <0^{g} |

NA, not applicable; ND, not done
^{A}0.05% Triton X-100 present
^{B}0.015% Triton X-100 present
^{C}5% impurity in D-isoleucyl analog due to D-allo-isoleucyl analog
^{D}cis-11 inhibits rFAAH only 13% at 1 μM, but inhibits hMAGL 66% at 100 nM
^{E}8 pt rFAAH IC₅₀ (95% confidence) JZL184 974 nM (784-1210) URB597 4.9 nM (4.1-6.0) n-C₁₆H₃₃SO₂F 6.3 nM (4.5-8.7) PMSF 833 nM (746-931) RHC80267 (1) 2,240 nM (2010-2500)
^{F}8 pt hMAGL IC₅₀ (95% confidence) JZL184 57 nM (53-62)
*protein was pretreated with JZL184 to completely inhibit MAGL activity

TABLE 2
Radio-TLC assay with [¹⁴C]SAG substrate of the inhibition of mDAGLα activity.

| | | mDAGLα | mDAGLα | mDAGLα | mDAGLα |
|---|---|---|---|---|---|
| | | inhibition | inhibition | inhibition | inhibition |
| Compound | Detail of N-formyl- | at 10 nM | at 100 nM | at 1000 nM | at 10000 nM |
| Number | α-amino ester | (%) | (%) | (%) | (%) |
| 3 (THL) | L-leucyl | 55 | 72 | 92 | 98 |
| 4 (OMDM-188) | L-isoleucyl | 69 | 80 | 96 | 96 |
| 5 | L-allo-isoleucyl | 60 | 61 | 88 | 100 |
| 6 | D-isoleucyl^{A} | 33 | 29 | 69 | 85 |
| 7 | D-allo-isoleucyl | 19 | 51 | 75 | 90 |
| 8 | (S)-α-aminobutyryl | 50 | 68 | 96 | 100 |
| 9 | NA | ND | ND | ND | <0 |
| JZL184 | NA | ND | ND | ND | 3 |
| URB597 | NA | ND | ND | ND | <0 |
| n-C₁₆H₃₃SO₂F | NA | ND | ND | 18 | 64 |
| JZL195 | NA | ND | ND | ND | 10 |

NA, not applicable;
ND, not done
^{A}5% impurity in D-isoleucyl analog due to D-allo-isoleucyl analog

TABLE 3
Results of in vitro FRET-based screening using reporter compound (17) of the
inhibition of lipase activities.

| | | hDAGLα | | |
|---|---|---|---|---|
| | | inhibition | Lipoprotein Lipase | Pancreatic Lipase |
| | | at 10 μM | (Bacterial) | (Porcine) |
| | | detergent free or | inhibition | inhibition |
| Compound | Detail of N-formyl- | (0.05% Triton X- | at 10 μM | at 10 μM |
| Number | α-amino ester | 100) (%) | (%) | (%) |
| 3 (THL) | L-leucyl | 92, 86° (99) | 92 | 96 |
| 4 (OMDM-188) | L-isoleucyl | 92 (99) | 84 | 99 |
| 5 | L-allo-isoleucyl | 92 (99) | 61 | 98 |
| 6 | D-isoleucyl^{A} | 90 (98) | 86 | 99 |
| 7 | D-allo-isoleucyl | 90 (98) | 68 | 95 |
| 8 | (S)-α-aminobutyryl | 93 (100) | 80 | 97 |
| 9 | none | 14 (72) | <0 | 36 |
| trans-10 | none | 69° | 67 | 96 |
| cis-11 | none | 65° | 33 | 94 |
| JZL184 | NA | 59, 44° | 36 | 36 |
| URB597 | NA | 22, 7° | 21 | 68 |
| n-C₁₆H₃₃SO₂F | NA | 40, 94° | 8 | 29 |
| PMSF | NA | 8° | <0 | 17 |
| RHC80267 (1) | NA | 70, 40° | <0 | 78 |
| SD41 | NA | 17, 7° | <0 | <0 |
| JZL195 | NA | 64, 55° | <0 | 43 |

NA, not applicable
^{A} 5% impurity in D-isoleucyl analog due to D-allo-isoleucyl analog
*protein was pretreated with JZL184 to completely inhibit MAGL activity

TLC DAGL assays with [¹⁴C]SAG substrate (or LC-MS assays with pure unlabeled SAG substrate) could result in significant errors if subsequent hydrolysis of 2-AG was not considered. It was advantageous that evaluations of enzymatic hydrolysis of [¹⁴C]SAG substrate include the sums of radiolabeled 2-AG and free radiolabeled arachidonic acid released. The crude cell preparations that were used had considerable monoacylglycerol lipase (MAGL), fatty acid amide hydrolase (FAAH), and other lipase activities which further degraded the radiolabeled 2-AG as it was formed under the assay conditions. It was very clear from the controls and from experiments with poor inhibitors that the release of labeled 2-AG was followed by further hydrolysis to labeled arachidonic acid. Thus DAGL activity was calculated via the sum of [¹⁴C]2-AG plus [¹⁴C]AA released divided by the sum of [¹⁴C]2-AG, [¹⁴C]AA, and final [¹⁴C]SAG concentrations for each lane. Also, DAGL activity in this human cell line (HEK293T) was not adjusted for hDAGLα and hDAGLβ activities demonstrated to be present in cell lysates following the mock infections.

The conversion of [¹⁴C]2-AG to [¹⁴C]AA was reduced in modified radio-TLC assays by pre-treatment of cell lysate with the highly selective MAGL inhibitor JZL 184 at 10 μM for 15 min. Then, tenfold dilution to 1 μM for screening assay use in some experiments as noted in the data tables gave little JZL 184 interference with hDAGLα activities in radioassays and fluorescent assays.

The use of 10 μM THL resulted in complete inhibition of (human) hDAGLα activity for all protein preparations (Table 1). Using TLC under basic conditions (J. Cell Biol. 2003, 163, 463 and Biochim. Biophys. Acta 2006, 1761, 205; each herein incorporated by reference in its entirety) and phosphorimaging analysis (J. Label. Compd. Radiopharm. 2009, 52, 324 and J. Org. Chem. 2011, 76, 2049; each herein incorporated by reference in its entirety), the radioassays consistently showed the apparent IC₅₀ of THL (3) to be in the range of 10 to 100 nM. Analogs (4)-(8) were also all extremely potent inhibitors of hDAGLα in radio-TLC assays. The β-lactones (9)-(11) and other compounds including JZL 184, PMSF, and RHC80267 (1) were poor inhibitors of hDAGLα. The β-lactone SD41 and ether lipid analogs of O-3841 synthesized did not inhibit hDAGLα. or mDAGLβ at 10 μM screening concentrations.

The β-lactones THL (3), OMDM-188 (4), and new analogs (5)-(9), were assayed for the inhibition of (murine) mDAGLα at 10 nM to 10 μM concentrations (Table 2) and FIG. 7. The D-isoleucyl- and D-alto-isoleucyl analogs (6) and (7) were clearly less potent than analogs of (S)-α-amino acids. Several analogs (THL (3), OMDM-188 (4), and the new L-allo analog (5) showed good selectivity for diacylglycerol lipase over monoacylglycerol lipase and fatty acid amide hydrolase enzymes. Future studies can include shorter alkyl chain analogs similar to the fatty acid synthase inhibitors from J. Med. Chem. 2008, 51, 5285 (herein incorporated by reference in its entirety) that might have improved solubility and membrane penetration properties.

### Example 4

### Lipid Substrates

Ether lipid substrates (17)-(22) (FIG. 2) for in vitro fluorescence resonance energy transfer (FRET) assay of DAGL and related lipase activities were developed. Lipase activity reporter molecules have been reported in, for example, Eur. J. Biochem. 1995, 231, 50; J. Lipid Res. 1996, 37, 868; J. Phys. Chem. B 1999, 103, 6680; Org. Biomol. Chem. 2006, 4, 1746; Tetrahedron Lett. 2008, 49, 3500; each herein incorporated by reference in its entirety. This series of novel ether lipid molecules was synthesized (Scheme 2) with the biomimetic stereochemistry at sn-2 and incorporated terminally functionalized sn-1 and sn-2 fatty acyl groups. The epoxide of (R)-(−)-glycidyl methyl ether (23) was opened with benzyl alcohol and sodium hydride followed by silyl protection of the secondary alcohol (24), and hydrogenolysis of the benzyl ether (25) in ethanol/ethyl acetate/acetic acid (1:1:0.2). Subsequent acylation of the primary hydroxyl group of (26), deprotection of the secondary alcohol t-butyldimethylsilyl group, followed immediately by acylation of the secondary hydroxyl of (28) gave ether lipid products (17)-(22) with only 5% impurity from acyl rearrangement.

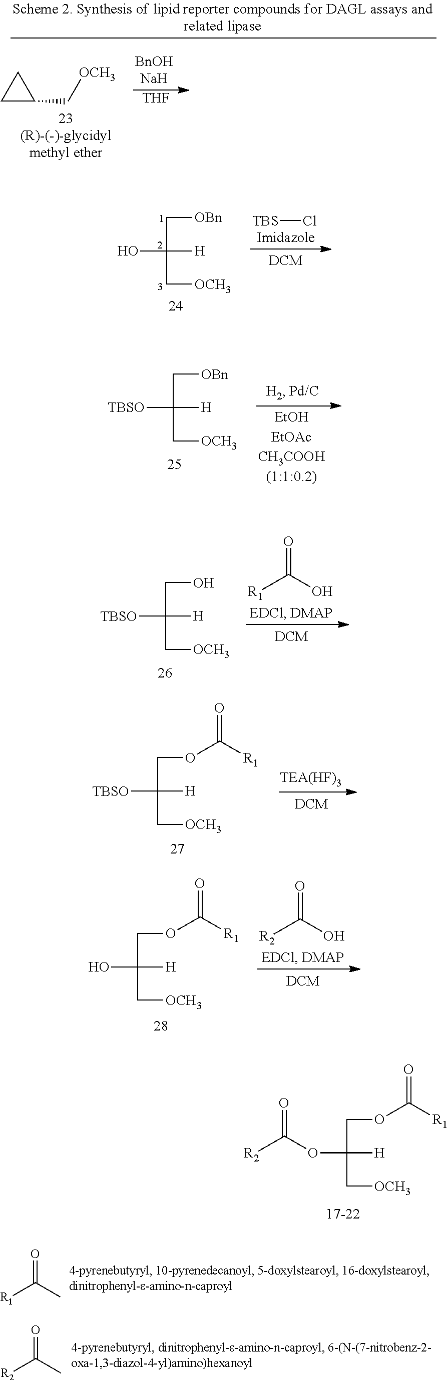

1-Benzyloxy-3-methoxy-sn-glycerol 24. Benzyl alcohol (2.69 g, 2.5 mL) was added drop-wise to a solution of NaH (0.59 g, 24.9 mmol) in THF at room temperature. The solution was brought to 0° C. before (R)-(−)-glycidyl methyl ether (2.0 g, 22.6 mmol) was added drop-wise. The mixture was stirred at room temperature overnight while being monitored by TLC (3:7 ethyl acetate/hexane: starting material Rf 0.10, product Rf 0.30). Upon completion, the mixture was concentrated and re-dissolved in CH₂Cl₂ (DCM) before being acidified using 1 M HCl. After a series of aqueous extractions were performed, the organic layers were dried with MgSO₄, filtered and concentrated. Purification by column chromatography (3:7 ethyl acetate/hexane) gave a 22% yield. ¹H NMR 63.39 (s, 3H), 3.42-3.59 (m, 4H), 3.96-4.05 (m, 1H), 4.57 (s, 2H), 7.28-7.53 (m, 5H).

1-Benzyl-2-tert-butyldimethylsilyl-3-methyl-sn-glycerol 25. TBDMS-Cl (0.168 g, 2.48 mmol) was added to a solution of secondary alcohol 24 (0.243 g, 1.24 mmol) and imidazole (0.168 g, 2.48 mmol) in DMF. The reaction was allowed to stir at room temperature for 12 hours while being monitored by TLC (5:95 ethyl acetate/hexane: starting material Rf 0.20, product Rf 0.85). Upon completion, an aqueous workup was performed. The resulting organic layers were dried with MgSO₄ and concentrated. Purification by column chromatography (5:95 ethyl acetate/hexane) gave a 66% yield. ¹H NMR δ 0.08 (d, J=2.93 Hz, 6H), 0.89 (s, 9H), 3.36 (s, 3H), 3.38 (dd, J=10.01, 5.62 Hz, 1H), 3.45 (m, J=8.79 Hz, 2H), 3.52 (m, J=5.37 Hz, 1H), 3.97 (quint, J=5.37 Hz, 1H), 4.55 (s, 2H), 7.28-7.42 (m, 5H).

2-tent-Butyldimethylsilyl-3-methyl-sn-glycerol 26. Benzyl-protected 25 (0.73 g, 2.36 mmol) was dissolved in a solution of ethanol, ethyl acetate and acetic acid (1:1:0.2). After the solution was degassed, 10% Pd/C (150 mg) was added. The solution was allowed to react with mechanical shaking overnight under hydrogen pressure (40 PSI). The reaction was monitored by TLC (1:4 ethyl acetate/hexane). Upon completion, the solution was filtered through a short pad of celite. Purification by column chromatography (1:4 ethyl acetate/hexane: product Rf 0.30) gave a quantitative yield. ¹H NMR δ 0.10 (s, 6H), 0.90 (s, 9H), 3.36 (s, 3H), 3.40 (m, J=5.86, 3.91 Hz, 2H), 3.58 (m, J=4.39 Hz, 1H), 3.64 (m, J=4.39 Hz, 1H), 3.83-3.92 (m, 1H).

1-((2,4-Dinitrophenyl)amino)hexanoyl-2-tert-butyldimethylsilyl-3-methyl-sn-glycerol 27a (—OC(O)R₁=dinitrophenyl-∈-amino-n-caproyl). Primary alcohol 26 (0.39 g, 1.77 mmol) was combined with DNP-∈-amino-n-caproic acid (0.578 g, 1.945 mmol) in dry CH₂Cl₂. The mixture was cooled to 0° C. before EDCI (0.848 g, 4.42 mmol) and DMAP (0.43 g, 3.54 mmol) were added. The solution was allowed to stir at room temperature for 12 hours while being monitored by TLC (1:4 ethyl acetate/hexane). Upon completion, an aqueous workup was performed and the organic layers were dried with MgSO₄, and concentrated under reduced pressure. Purification by column chromatography (1:4 ethyl acetate/hexane: product Rf 0.20) gave an 80% yield. ¹H NMR δ 0.08 (d, J=2.44 Hz, 6H), 0.88 (s, 9H), 1.26 (t, J=7.32 Hz, 2H), 1.50 (m, J=7.32 Hz, 2H), 1.72 (quint, J=7.57 Hz, 2H), 1.81 (quint, J=7.45 Hz, 2H), 2.37 (t, J=7.32 Hz, 2H), 3.35 (s, 3H), 3.42 (m, J=5.37 Hz, 2H), 3.93-4.05 (m, 2H), 4.08-4.24 (m, 1H), 6.91 (d, J=9.77 Hz, 1H), 8.28 (dd, J=9.28, 2.44 Hz, 1H), 8.50-8.62 (m, 1H), 9.16 (d, J=2.44 Hz, 1H).

1((2,4-Dinitrophenyl)amino)hexanoyl-3-methyl-sn-glycerol 28a (—OC(O)R₁=dinitrophenyl-∈-amino-n-caproyl). Tetrabutylammonium fluoride (1.69 mmol, 1.69 mL) was added drop-wise to a solution of silyl-protected 1-((2,4-dinitrophenyl)amino)hexanoyl-2-tert-butyldimethylsilyl-3-methyl-sn-glycerol 27a (0.73 g, 1.45 mmol) in THF. The solution was allowed to stir at room temperature under nitrogen for 3 hours while being monitored by TLC (1:1 ethyl acetate/hexane). Following concentration, purification by column chromatography (1:1 ethyl acetate/hexane: product Rf 0.15) gave a 79% yield. ¹F1 NMR δ 1.48-1.61 (m, 4H), 1.70-1.78 (m, 2H), 1.82 (quint, J=7.32 Hz, 2H), 2.39-2.47 (m, 2H), 3.41 (s, 3H), 3.42-3.51 (m, 2H), 3.97-4.05 (m), 4.13 (m, 1H), 4.17-4.23 (m, 1H), 6.92 (d, J=9.28 Hz, 1H), 8.29 (dd, J=9.28, 2.44 Hz, 1H), 8.56 (br s, 1H), 9.16 (d, J=2.44 Hz, 1H).

1-((2,4-Dinitrophenyl)amino)hexanoyl-2-pyrenebutanoyl-3-methyl-sn-glycerol 17. 1-Pyrene butyric acid (31.4 mg, 0.108 mmol) was added to 1-((2,4-dinitrophenyl)amino)hexanoyl-3-methyl-sn-glycerol 28a (35.0 mg, 0.0908 mmol) in dry DCM. The solution was magnetically stirred and kept under nitrogen. The reaction mixture was cooled to 0° C. before EDCI (60.9 mg, 0.317 mmol) and DMAP (22.0 mg, 0.181 mmol) were added. The solution was allowed to react for several hours while being monitored by TLC (1:1 ethyl acetate/hexane: starting material Rf 0.40, product Rf 0.55). After consumption of starting material, the reaction mixture was subjected to an aqueous workup and extraction with DCM. The organic layer was dried with MgSO₄ and concentrated under reduced pressure. Purification by column chromatography (4:6 ethyl acetate/hexane) gave a 44% yield of 1-DNP-2-pyrenyl ether lipid 17 as a viscous yellow semi-solid. ¹H NMR δ 1.20-1.32 (m, 2H), 1.46 (quintet, J=7.5 Hz, 2H), 1.59 (quint, J=7.6 Hz, 2H), 2.14-2.26 (m, 2H), 2.30 (t, J=7.3 Hz, 2H), 2.54 (t, J=7.1 Hz, 2 HI, 2.89 (apparent q, J=7.3 Hz, 2H), 3.39 (s, 3H), 3.34-3.44 (m, 2H), 3.55 (dd, J=10.7, 5.1 Hz, 1H), 3.58 (dd, J=10.7, 5.1 Hz, 1H), 4.22 (dd, J=12.2, 7.3 Hz, 1H), 4.44 (dd, J=12.0, 3.2 Hz, 1H), 5.31-5.38 (m, 1H), 6.37 (d, J=9.8 Hz, 1H), 7.85 (d, J=7.8 Hz, 1H), 7.90-8.01 (m, 4H), 8.05-8.18 (m, 5H), 8.28 (d, J=9.3 Hz, 1H), 8.89 (d, J=2.9 Hz, 1H).

1-Pyrenebutanoyl-2-tert-butyldimethylsilyl-3-methyl-sn-glycerol 27b (OC(O)R₁=4-pyrenebutyryl). Primary alcohol 26 (67.6 mg, 0.3 mmol) was combined with 1-pyrenebutyric acid (0.106 g, 0.37 mmol) in dry CH₂Cl₂. The mixture was cooled to 0° C. before EDCI (14.6 mg, 0.77 mmol) and DMAP (7.4 mg, 0.61 mmol) were added. The solution was allowed to stir at room temperature for 12 hours while being monitored by TLC (3:7 ethyl acetate/hexane). Upon completion, an aqueous workup was performed and the organic layers were dried with MgSO₄ and concentrated under reduced pressure. Purification by column chromatography (3:7 ethyl acetate/hexane: product Rf 0.75) gave a 66% yield. ¹H NMR δ 0.05 (d, J=6.35 Hz, 6H), 0.85 (s, 9H), 2.19 (quint, J=7.45 Hz, 4H), 2.46 (t, J=7.32 Hz, 2H), 3.33 (s, 3H), 3.33-3.42 (m, 2H), 3.93-4.00 (m, 1H), 4.02 (m, J=6.35 Hz, 1H), 4.20 (dd, J=11.23, 3.91 Hz, 1H), 7.84 (d, J=7.81 Hz, 1H), 7.93-8.04 (m, 3H), 8.05-8.11 (m, 2H), 8.14 (t, J=6.59 Hz, 2H), 8.28 (d, J=9.28 Hz, 1H).

1-Pyrenebutanoyl-3-methyl-sn-glycerol 28b (OC(O)R₁=4-pyrenebutyryl). Triethylamine trihydrofluoride (48.6 mg, 3.0 mmol) was added to a solution of silyl-protected 1-pyrenebutanoyl-2-tert-butyldimethylsilyl-3-methyl-sn-glycerol 27b (99.5 mg, 0.2 mmol) in dry CH₂Cl₂. The solution was allowed to react for 48 hours at room temperature while being monitored by TLC (1:1 ethyl acetate/hexane). Upon completion, the reaction mixture was concentrated and utilized directly in the next step.

1-Pyrenebutanoyl-2-((2,4-dinitrophenyl)amino)hexanoyl-3-methyl-sn-glycerol 18. DNP-∈-amino-n-caproic acid (71.6 mg, 0.24 mmol) was added to 1-pyrenebutanoyl-3-methyl-sn-glycerol 28b (76.0 mg, 0.2 mmol) in dry DCM. The solution was magnetically stirred and kept under nitrogen. The reaction mixture was cooled to 0° C. before EDCI (134 mg, 0.7 mmol) and DMAP (48.8 mg, 0.4 mmol) were added. The solution was allowed to react for several hours while being monitored by TLC (1:1 ethyl acetate/hexane: starting material Rf 0.40, product Rf 0.55). After consumption of starting material, the reaction mixture was subjected to an aqueous workup and extraction with DCM. The organic layer was dried with MgSO₄ and concentrated under reduced pressure. Purification by column chromatography (4:6 ethyl acetate/hexane) gave a 50% yield of 1-pyrenyl-2-DNP ether lipid 18 as a viscous yellow semi-solid. ¹H NMR δ 1.21-1.30 (m, 2H), 1.44 (quintet, J=7.4 Hz, 2H), 1.58 (quintet, J=7.6 Hz, 2H), 2.18 (quintet, J=7.4 Hz, 2H), 2.32 (t, J=7.3 Hz, 2H), 2.48 (t, J=7.3 Hz, 2H), 2.83 (dd, J=13.2, 7.3 Hz, 2H), 3.36 (s, 3H), 3.32-3.38 (m, 2H), 3.52 (dd, J=10.7, 5.4 Hz, 2H), 3.54 (dd, J=10.7, 5.4 Hz, 2H), 4.21 (dd, J=12.0, 6.6 Hz, 1H), 4.43 (dd, J=12.2, 3.4 Hz, 1H), 5.23-5.30 (m, 1H), 6.28 (d, J=9.3 Hz, 1H), 7.82 (d, J=7.3 Hz, 1H), 7.85 (dd, J=9.5, 2.7 Hz, 1H), 7.90-7.97 (m, 3H), 8.02-8.12 (m, 5H), 8.23 (d, J=9.3 Hz, 1H), 8.83 (d, J=2.4 Hz, 1H). HRMS for C₃₆H₃₇N₃O₉ [MH⁺] calc'd, 655.25183; found, 655.25293.

1-Pyrenedecanoyl-2-tert-butyldimethylsilyl-3-methyl-sn-glycerol 27c (OC(O)R₁=10-pyrenedecanoyl). Primary alcohol 26 (13.4 mg, 0.06 mmol) was combined with 1-pyrenedecanoic acid (25.0 mg, 1.27 mmol) in dry CH₂Cl₂. The mixture was cooled to 0° C. before EDCI (30.0 mg, 2.89 mmol) and DMAP (14.9 mg, 0.12 mmol) were added. The solution was allowed to stir at room temperature for 12 hours while being monitored by TLC (1:9 ethyl acetate/hexane). Upon completion, an aqueous workup was performed and the organic layers were dried with MgSO₄ and concentrated under reduced pressure. Purification by column chromatography (1:9 ethyl acetate/hexane) gave a quantitative yield. ¹H NMR δ 0.10 (s, 6H), 0.90 (s, 9H), 1.32 (br s, 8H), 1.35-1.43 (m, 2H), 1.49 (quint, J=7.32 Hz, 2H), 1.63 (m, J=6.84 Hz, 2H), 1.86 (quint, J=7.69 Hz, 2H), 2.32 (t, J=7.57 Hz, 2H), 3.32-3.35 (m, 1H), 3.36 (s, 3H), 3.37-3.40 (m, 1H), 4.01 (d, J=6.35 Hz, 2H), 4.19 (q, J=6.84 Hz, 1H), 7.88 (d, J=7.81 Hz, 1H), 7.96-8.07 (m, 3H), 8.12 (dd, J=8.55, 4.15 Hz, 2H), 8.17 (m, J=5.37 Hz, 2H), 8.29 (d, J=9.28 Hz, 1H).

1-Pyrenedecanoyl-3-methyl-sn-glycerol 28c (OC(O)R₁=10-pyrenedecanoyl). Triethylamine trihydrofluoride (0.152 g, 0.95 mmol) was added to a solution of silyl-protected 1-pyrenedecanoyl-2-tert-butyldimethylsilyl-3-methyl-sn-glycerol 27c (36.4 mg, 0.063 mmol) in dry CH₂Cl₂. The solution was allowed to react for 48 hours at room temperature while being monitored by TLC (1:1 ethyl acetate/hexane). Upon completion, the reaction mixture was concentrated and utilized directly in the next step.

1-Pyrenedecanoyl-2-((2,4-dinitrophenyl)amino)hexanoyl-3-methyl-sn-glycerol 19. DNP-∈-amino-n-caproic acid (23.1 mg, 0.078 mmol) was added to alcohol 1-pyrenedecanoyl-3-methyl-sn-glycerol 28c (30 mg, 0.06 mmol) in dry DCM. The solution was magnetically stirred and kept under nitrogen. The reaction mixture was cooled to 0° C. before EDCI (40 mg, 0.21 mmol) and DMAP (14.6 mg, 0.12 mmol) were added. The solution was allowed to react for several hours while being monitored by TLC (1:1 ethyl acetate/hexane: starting material Rf 0.50, product Rf 0.70). After consumption of starting material, the reaction mixture was subjected to an aqueous workup and extraction with DCM. The organic layer was dried with MgSO4 and concentrated under reduced pressure. Purification by column chromatography (3:7 ethyl acetate/hexane) gave a 46% yield. ¹H NMR δ 1.31 (br s, 6H), 1.35-1.43 (m, 2H), 1.42-1.55 (m, 2H), 1.54-1.65 (m, 4H), 1.64-1.75 (m, 4H), 1.86 (quint, J=7.57 Hz, 2H), 2.31 (t, J=7.32 Hz, 2H), 2.38 (t, J=7.32 Hz, 2H), 3.21 (dd or q, J=12.21 Hz, 6.84 Hz, 2H), 3.33 (t, 2H), 3.37 (s, 3H), 3.53 (dd, J=4.88, 1.95 Hz, 2H), 4.15 (dd, J=11.72, 6.35 Hz, 1H), 4.36 (dd, J=12.21, 3.91 Hz, 1H), 5.23 (dt, J=10.25 Hz, 5.37 Hz, 1H), 6.67 (d, J=9.28 Hz, 1H), 7.86 (d, J=7.81 Hz, 1H), 7.94-8.04 (m, 4H), 8.06-8.18 (m, 4H), 8.26 (d, J=9.28 Hz, 1H), 8.39 (m, 1H), 9.03 (d, J=2.44 Hz, 1H). HRMS for C₄₂H₄₉N₃O₉ [MH⁺] calc'd, 739.34683; found, 739.34693.

1-((2,4-Dinitrophenyl)amino)hexanoyl-2-(N-(7-nitrobenz-2-oxa-1,3-diazol-4-yl)amino)hexanoyl-3-methoxy-sn-glycerol 20. 6-(N-(7-Nitrobenz-2-oxa-1,3-diazol-4-yl)amino)hexanoic acid (36.6 mg, 0.12 mmol) was added to 1-((2,4-dinitrophenyl)amino)hexanoyl-3-methyl-sn-glycerol 28a (40 mg, 0.10 mmol) in dry DCM. The solution was magnetically stirred and kept under nitrogen. The reaction mixture was cooled to 0° C. before EDCI (69.0 mg, 0.36 mmol) and DMAP (25.16 mg, 0.20 mmol) were added. The solution was allowed to react for several hours while being monitored by TLC (1:1 ethyl acetate/hexane: starting material Rf 0.40, product Rf 0.55). After consumption of starting material, the reaction mixture was subjected to an aqueous workup and extraction with DCM. The organic layer was dried with MgSO₄ and concentrated under reduced pressure. Purification was by column chromatography (4:6 ethyl acetate/hexane). ¹H NMR δ 1.52 (m, 4H), 1.65-1.77 (m, 4H), 1.75-1.88 (m, 4H), 2.38 (dt, J=15.02, 7.39 Hz, 4H), 3.36 (s, 3H), 3.44 (dd, J=12.21, 6.84 Hz, 2H), 3.48-3.59 (m, 4H), 4.14 (dd, J=11.96, 6.10 Hz, 1H), 4.38 (dd, J=12.21, 3.91 Hz, 1H), 5.22 (ddd, J=10.25 Hz, 5.37 Hz, 1H), 6.18 (d, J=8.30 Hz, 1H), 6.57 (t, J=5.13 Hz, 1H), 6.92 (d, J=9.28 Hz, 1H), 8.26 (dd, J=9.52, 2.69 Hz, 1H), 8.47 (d, J=8.79 Hz, 1H), 8.52-8.60 (m, 2H), 9.10 (d, J=2.44 Hz, 1H). HRMS for C₂₈H₃₅N₇O₁₂ [MH⁺] calc'd, 661.23303; found, 661.23433.

1-(5-Dimethyloxazolinyloxyl)stearoyl-2-tert-butyldimethylsilyl-3-methyl-sn-glycerol 27d (OC(O)R¹=5-doxylstearoyl). Primary alcohol 26 (6.0 mg, 0.027 mmol) was combined with 5-DOXYL-stearic acid (10.5 mg, 0.027 mmol) in dry CH₂Cl₂. The mixture was cooled to 0° C. before EDCI (13.0 mg, 0.07 mmol) and DMAP (6.6 mg, 0.054 mmol) were added. The solution was allowed to stir at room temperature for 12 hours while being monitored by TLC (2:8 ethyl acetate/hexane). Upon completion, an aqueous workup was performed and the organic layers were dried with MgSO₄ and concentrated under reduced pressure. Purification by column chromatography (2:8 ethyl acetate/hexane: product Rf 0.70) gave a 68% yield.

1-(5-Dimethyloxazolinyloxyl)stearoyl-3-methyl-sn-glycerol 28d. Triethylamine trihydrofluoride (28.3 mg, 0.175 mmol) was added to a solution of silyl-protected 1-(5-dimethyloxazolinyloxyl)stearoyl-2-tert-butyldimethylsilyl-3-methyl-sn-glycerol 27d (10.3 mg, 0.0175 mmol) in dry CH₂Cl₂. The solution was allowed to react for 48 hours at room temperature while being monitored by TLC (3:7 ethyl acetate/hexane: product Rf 0.15). Upon completion, the reaction mixture was concentrated and utilized directly in the next step.

1-(5-Dimethyloxazolinyloxyl)stearoyl-2-pyrenebutanoyl-3-methyl-sn-glycerol 21 1-Pyrenebutyric acid (16.1 mg, 0.06 mmol) was added to alcohol 1-(5-dimethyloxazolinyloxyl)stearoyl-3-methyl-sn-glycerol 28d (22 mg, 0.046 mmol) in dry DCM. The solution was magnetically stirred and kept under nitrogen. The reaction mixture was cooled to 0° C. before EDCI (31 mg, 0.16 mmol) and DMAP (11.4 mg, 0.09 mmol) were added. The solution was allowed to react for several hours while being monitored by TLC (1:4 ethyl acetate/hexane: starting material Rf 0.15, product Rf 0.40). After consumption of starting material, the reaction mixture was subjected to an aqueous workup and extraction with DCM. The organic layer was dried with MgSO₄ and concentrated under reduced pressure. Purification was by column chromatography (1:5 ethyl acetate/hexane). HRMS for C₄₆H₆₆NO₇. [MH⁺] calc'd, 744.44840; found, 744.4839.

1-(16-Dimethyloxazolinyloxyl)stearoyl-2-tert-butyldimethylsilyl-3-methyl-sn-glycerol 27e (OC(O)R¹=16-doxylstearoyl). Primary alcohol 26 (13.0 mg, 0.059 mmol) was combined with 16-DOXYL-stearic acid (25.0 mg, 0.06 mmol) in dry CH₂Cl₂. The mixture was cooled to 0° C. before EDCI (28.2 mg, 0.147 mmol) and DMAP (17.9 mg, 0.147 mmol) were added. The solution was allowed to stir at room temperature for 12 hours while being monitored by TLC (2:8 ethyl acetate/hexane: product Rf 0.70). Upon completion, an aqueous workup was performed and the organic layers were dried with MgSO₄ and concentrated under reduced pressure. Purification by column chromatography (5:95 ethyl acetate/hexane) gave a 33% yield.

1-(16-Dimethyloxazolinyloxyl)stearoyl-3-methyl-sn-glycerol 28e (OC(O)R¹=16-doxylstearoyl). Triethylamine trihydrofluoride (31.6 mg, 0.195 mmol) was added to a solution of silyl-protected 1-(16-dimethyloxazolinyloxyl)stearoyl-2-tert-butyldimethylsilyl-3-methyl-sn-glycerol 27e (11.5 mg, 0.0195 mmol) in dry CH₂Cl₂. The solution was allowed to react for 48 hours at room temperature while being monitored by TLC (3:7 ethyl acetate/hexane: product Rf 0.15). Upon completion, the reaction mixture was concentrated and utilized directly in the next step.

1-(16-Dimethyloxazolinyloxyl)stearoyl-2-pyrenebutanoyl-3-methyl-sn-glycerol 22. 2-Pyrenebutanoic acid (6.58 mg, 0.02 mmol) was added to alcohol 1416-dimethyloxazolinyloxyl)stearoyl-3-methyl-sn-glycerol 28e (9.0 mg, 0.019 mmol) in dry DCM. The solution was magnetically stirred and kept under nitrogen. The reaction mixture was cooled to 0° C. before EDCI (12.7 mg, 0.06 mmol) and DMAP (4.6 mg, 0.03 mmol) were added. The solution was allowed to react for several hours while being monitored by TLC (1:4 ethyl acetate/hexane: starting material Rf 0.15, product Rf 0.40). After consumption of starting material, the reaction mixture was subjected to an aqueous workup and extraction with DCM. The organic layer was dried with MgSO₄ and concentrated under reduced pressure. Purification by column chromatography (1:5 ethyl acetate/hexane) gave a 40% yield. HRMS for C₄₆H₆₆NO₇. [MH⁺] calc'd, 744.4839; found, 744.4844.

Alternatively, other protected glycidyl building blocks can be used wherein the methyl ether of (23) is replaced with a suitable group.

The FRET pairs initially studied were the pyrene and nitrobenzoxadiazole (NBD) fluorophors with either the dinitrophenyl or nitroxyl group quenchers. Excitation of the pyrene or NBD results in radiationless energy transfer to the quenchers when close enough and sufficiently well oriented. The fully extended distances between the fluorophors and quenchers were estimated (Schrodinger Suite 2010, in an aqueous environment with a dielectric constant of 80) to be 18 angstroms for (17), (18), and the NBD analog (20); and, to be 24 angstroms for the pyrenedecanoyl analog (19). The assays with NBD analog (20) had too much baseline instability as this fluorescent group is quite sensitive to the polarity of its environment

The pyrene was estimated to be 15 angstroms and 24 angstroms from the nitroxyl stable free radical quenching groups (Biophys. J. Biophys. Letters 2005, 89, L37; herein incorporated by reference in its entirety) of the 5-doxylstearoyl analog (21) and the 16-doxylstearoyl analog (22), respectively, and both compounds were poor substrates for enzymatic hydrolysis. The pyrene-dinitrophenyl FRET pairings (17) and (18) were stable to uncatalyzed hydrolysis at neutral pH, were the best enzyme substrates, and were readily utilized in a 96-well format. A convenient in vitro fluorometric esterase assay utilizing the BioTek Instruments Synergy™ HT Multi-Mode Microplate 96-well reader was developed that measured nanomolar concentrations of fluorescent reaction product.

### Example 5

### In Vitro FRET-Based DAGL Assays

Both configurations of the FRET pairing of pyrene donor and dinitrophenyl acceptor (17) and (18) were satisfactory, though the 2-pyrenyl analog (17) was used for all in vitro FRET assays. The fluorescent assays were run in the same Tris buffer with calcium used for the radiochemical assays except that 200 μL final volumes were needed for efficient reading. The lipoprotein lipase standard (0.23 μg) was again used for a positive control. The freshly sonicated HEK cell lysate (100 μg total) protein containing DAGL was used as a suspension for each assay. A 15 min period of gentle shaking at ambient temperature was used following the addition of 10 μL of pure DMSO (for the control) or the 10 μL DMSO solutions of inhibitors. The ether lipid substrate (25 μM final concentration) was then added in DMSO (10 μL) to all wells, and after 2 min of shaking at 37° C., an initial reading was taken with excitation at 320 nm and emission observed at 400 nm. Every 14 min, another 1 min of shaking would precede the fluorescence readings. The readings were followed over 2 h, but the timepoint of 1.5 h was used to calculate percent inhibitions. The inhibition of hydrolysis of the ether lipid reporter compound (17) by the DAGL-containing protein preparations with the compounds under investigation screened at 10 μM was then compared with 10 μM THL control, which gives complete inhibition of all human and murine DAGLs tested with the radiolabeled endogenous substrate [¹⁴C]SAG. Using the pyrene-dinitrophenyl reporter compound (17), the apparent IC₅₀ of THL was always approximately 10 nM with hDAGLα using this in vitro FRET-based assay.

Cell lysate or membrane preparations containing overexpressed DAGL catalyzed the hydrolysis of the reporter substrate, and a fluorescence response increased at a nearly linear rate for over two hours. Wells that had a 15 minute pre-incubation with DAGL inhibitors and that showed a concentration dependent attenuation of fluorescence response were identified as “hits.” At a screening concentration of 10 μM, compounds (3)-(8) were identified as potent inhibitors of hDAGLα (Table 3). However, any potential DAGL inhibitor identified from the in vitro FRET assay should then be submitted to the TLC assay with the radiolabeled endogenous diglyceride substrate to identify any false positives for DAGL inhibition. Fluorescence results could reflect inhibition of other hydrolytic enzymes in the crude cell preparations that hydrolyze the ether lipid substrates (17)-(22) to a much greater extent than the 1,2-diacyl-sn-glycerol substrate. Using highly selective enzyme inhibitors JZL 184, UR8597, and others, the enzymes responsible for false positives include monoacylglycerollipase (MGL) and fatty acid amide hydrolase (FAAH). The ether lipid FRETsubstrate (17) will be most useful for assays with DAGLs purified to homogeneity.

Additional assays were performed to establish the selectivity of DAGL inhibitors. Compounds (3)-(11) were assayed for binding to the CB1 (rat brain preparation) and CB2 (mouse or human receptor expressed in HEK293), and none had a Ki below 1 μM in these competition binding experiments.

### Example 6

### Cannabinoid Receptor Binding

Assays were performed by the methods reported in J. Med. Chem. 2007, 50, 6493 and J. Med. Chem. 2008, 51, 6393; each herein incorporated by reference in its entirety. Palmitylsulfonyl fluoride (n-C₁₆H₃₃SO₂F) had an apparent IC₅₀ of 440 nM, which correlates well with the literature report for rCB1 (520 nM; J. Med. Chem. 2008, 51, 6393; herein incorporated by reference in its entirety). All other standards (including RHC80267 and SD41) did not bind to the cannabinoid receptors, analogous to the previous reports for THL(3), JZL 184, URB597, and PMSF (J. Physiol. 2006, 577, 263; J. Med. Chem. 2008, 51, 6970; Nat. Chem. Biol. 2009, 5, 37; Nat. Med. 2003, 9, 76; CNS Drug Rev. 2006, 12, 21; Biochem. Biophys. Res. Commun. 1997, 231, 217; each herein incorporated by reference in its entirety).

Compounds (3)-(11) were also assayed for inhibition of endocannabinoid hydrolytic enzymes in fluorescence-based assays (Table 1). Assays of the inhibition of fatty acid amide hydrolase (rFAAH) used the reported coumarin amide reporter compound (Anal. Biochem. 2005, 343, 143; herein incorporated by reference in its entirety).

### Example 7

### Inhibition of rat FAAH

The N-terminal his-tagged rFAAH deletion sequence used was expressed in an E. coli cell line provided by the Cravatt group (Biochemistry 1998, 37, 15177; herein incorporated by reference in its entirety). The rFAAH coumarin ester substrate fluorescence assay demonstrated UR8597 to have an apparent IC₅₀ of 4.9 nM. This is comparable to inhibition of rat membrane preparations used for the hydrolysis of tritiated anandamide (Nat. Med. 2003, 9, 76; J. Med. Chem. 2004, 47, 4998; Pharmacolog. Res. 2006, 54, 481; each herein incorporated by reference in its entirety). Palmitylsulfonyl fluoride (n-C₁₆H₃₃SO₂F) had an apparent IC₅₀ of 6.3 nM in the fluorescent rFAAH assay (approximately 2 μM in the hMAGL assay detailed below) which correlates well with the IC₅₀ of 7 nM using the radiolabeled N-arachidonoylethanolamine (anandamide) substrate (Biochem. Biophys. Res. Commun. 1997, 231, 217; herein incorporated by reference in its entirety). All other standards THL (3), JZL 184, PMSF, and RHC80267 have been reported to be poor inhibitors of FAAH activity (J. Physiol. 2006, 577, 263; Bioorg. Med. Chem. Lett. 2008, 18, 5838; J. Med. Chem. 2008, 51, 6970; Nat. Chem. Biol. 2009, 5, 37; Biochem. Biophys. Res. Commun. 1997, 231, 217; Biochem. Pharmacol. 1997, 53, 255; each herein incorporated by reference in its entirety).

Assays of the inhibition of monoacylglycerollipase (hMGL) used the 7-hydroxy-6-methoxy analog (below and J. Proteome Res. 2008, 7, 2158; herein incorporated by reference in its entirety) of the reported coumarin ester (Assay Drug. Dev. Technol. 2008, 6, 387; herein incorporated by reference in its entirety).

### Example 8

### Inhibition of human MAGL

The N-terminal his-tagged full length human monoacylglycerol lipase used was expressed in E. coli (J. Proteome Res. 2008, 7, 2158; herein incorporated by reference in its entirety). The coumarin substrate fluorescence assay demonstrated JZL 184 to have an apparent IC₅₀ of 57 nM that is comparable to human recombinant MAGL expressed in COST cells where the IC₅₀ of JZL 184 was reported to be 2 to 6 nM with the endogenous substrate 2-AG (Nat. Chem. Biol. 2009, 5, 37; Chem. Biol. 2009, 16, 744; each herein incorporated by reference in its entirety). All other standards, THL (3), JZL 184, URB597, palmitylsulfonyl fluoride (n-C₁₆H₃₃SO₂F), PMSF, and RHC80267 were reported to be poor inhibitors of MAGL activity (J. Physiol. 2006, 577, 263; Biochim. Biophys. Acta 2006, 1761, 205; Bioorg. Med. Chem. Lett. 2008, 18, 5838; J. Med. Chem. 2008, 51, 6970; Nat. Chem. Biol. 2009, 5, 37; Nat. Med. 2003, 9, 76; Biochem. Pharmacol. 2004, 67, 1381; each herein incorporated by reference in its entirety).

The assays were validated with standard compounds including the selective FAAH inhibitor URB597 (J. Med. Chem. 2004, 47, 4998; herein incorporated by reference in its entirety) and the selective MAGL inhibitor JZL 184 (Nat. Chem. Biol. 2009, 5, 37; herein incorporated by reference in its entirety). Preliminary studies with [1″-¹⁴C]SAG using the purified rFAAH and hMAGL endocannabinoid hydrolytic enzymes in these fluorescent assays showed low activities of these enzymes for the 1,2-diglyceride substrate. Also, preliminary studies with ether lipid substrate (17) in the in vitro FRET-based assay confirmed the potent inhibition by THL (3) (supra, also see Biochemistry 1998, 37, 15177; herein incorporated by reference in its entirety) of commercially available homogeneous bacterial lipoprotein lipase and porcine triacylglycerol lipase enzymes (Table 3).

### Example 9

### Other Novel In Vitro FRET-Based Assays

The bacterial lipoprotein lipase assays (0.46 μg of high specific activity lipase as described above) and the porcine pancreatic lipase type II assays (1.0 μg suspended in 1:9 DMSO/water, Sigma L3126, labeled 100-400 units/mg with olive oil substrate) also used pyrene-dinitrophenol reporter compound (17) (25 μM in 200 μL volumes), which was a better substrate than the isomeric (18). The apparent IC₅₀ of THL was approximately 100 nM with lipoprotein lipase, but less than 1 nM with the porcine pancreatic lipase using this FRET assay.

This new FRET-based methodology should be suitable for the assay of new inhibitors of human recombinant proteins including lipoprotein lipase, triacylglycerol lipase, and other related hydrolases to determine DAGL selectivity in vitro.

In summary, structure-activity relationship (SAR) studies have demonstrated molecular features of inhibitors that result in inactivation of human and murine DAGLs at nanomolar inhibitor concentrations. The importance of a small (S)—N-formyl-α-amino group as a structural feature in targeting DAGLs was clearly demonstrated. The (3-lactone was the most active of the covalently reactive quasi-irreversible inactivating functional groups that were tested for DAGL inhibition. Many factors affect assay results including protein concentration, substrate structure and concentration, length of the incubation period for enzyme inactivation, and the presence of co-solvent and detergent. An in vitro FRET-based screen was established for rapidly identifying inhibitors of DAGL activity. Although false positives can occur due to inhibition of other hydrolytic enzymes present in the cell lysate or membrane preparations used, the assay will be suitable for the preliminary screening of compound libraries. An improved and detailed radio-TLC assay of DAGL activity with the labeled endogenous substrate [1″-¹⁴C]1-stearoyl-2-arachidonoyl-sn-glycerol was utilized to unambiguously distinguish DAGL activity from the activities of MAGL and FAAH. Thus, methodologies were established to determine α/β-subtype selectivity as well as selective inhibition of DAGL over esterases, amidases, and other lipases.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

1. A compound of formula (I), 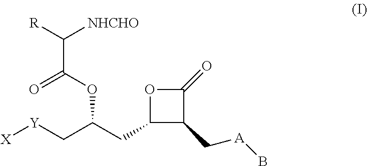
wherein,
R is (C₁-C₁₂)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is a solid support or H;
X is a solid support or H;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100; or a pharmaceutically acceptable salt thereof.

2. The compound of claim 1, wherein at least one of B or X is a solid support.

3. The compound of claim 1, wherein
R is (C₁-C₆)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is a solid support or H;
X is a solid support or H, wherein at least one of B or X is a solid support;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100.

4. The compound of claim 1, wherein
R is (C₁-C₆)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is H;
X is a solid support;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100.

5. The compound of claim 1, wherein
R is (C₁-C₆)-alkyl or (C₆-C₁₂)-aryl;
A is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is H;
X is a solid support;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100.

6. The compound of claim 1, wherein
R is (C₁-C₄)-alkyl;
A is (C₁-C₁₂)-alkyl;
B is H;
X is a solid support;
Y is a linking group comprising -J-, —O-J-, or —S-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
n is 0-10.

7. The compound of claim 1, wherein
R is (C₁-C₄)-alkyl;
A is (C₁-C₁₂)-alkyl;
B is H;
X is polyvinyl chloride or cellulose;
Y is a linking group comprising -J-, —O-J-, or —S-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
n is 0-10.

8. A compound of formula (II), 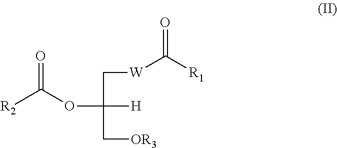
wherein,
W is O, NH, or N—(C₁-C₃)-alkyl;
R₁ is (C₁-C₁₂)-alkyl; (C₁-C₁₂)-alkyl-aryl, wherein aryl is optionally substituted with one or more nitro groups; (C₁-C₁₂)-alkyl-NH-aryl, wherein aryl is optionally substituted with one or more nitro groups; —NH(C₁-C₈)-alkyl, —O(C₁-C₈)-alkyl, —NH(C₁-C₈)-alkyl, 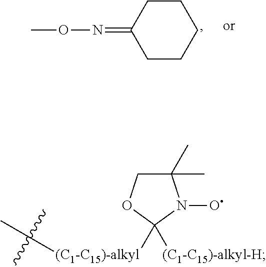
R₂ is (C₁-C₂₀)-alkyl; (C₁-C₂₀)-alkenyl; (C₁-C₂₀)-alkyl-aryl, wherein aryl is optionally substituted with one or more nitro groups; (C₁-C₂₀)-alkyl-NH-aryl, wherein aryl is optionally substituted with one or more nitro groups; (C₁-C₂₀)-alkyl-heteroaryl, wherein heteroaryl is optionally substituted with one or more nitro groups; or —NH(C₁-C₈)-alkyl; and
R₃ is H or (C₁-C₁₂)-alkyl; or a pharmaceutically acceptable salt thereof.

9. The compound of claim 8, wherein
W is O, NH, or N—(C₁-C₃)-alkyl; 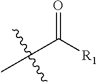
is 4-pyrenebutyryl, 10-pyrenedecanoyl, 5-doxylstearoyl, 16-doxylstearoyl, or dinitrophenyl-∈-amino-n-caproyl; 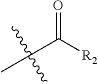
is 4-pyrenebutyryl, dinitrophenyl-∈-amino-n-caproyl, or 6-(N-(7-nitrobenz-2-oxa-1,3-diazol-4-yl)amino)hexanoyl; and
R₃ is H or (C₁-C₂)-alkyl.

10. The compound of claim 9, wherein
W is O; 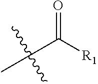
is 4-pyrenebutyryl, 10-pyrenedecanoyl, 5-doxylstearoyl, 16-doxylstearoyl, or dinitrophenyl-∈-amino-n-caproyl; 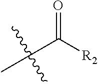
is 4-pyrenebutyryl, dinitrophenyl-∈-amino-n-caproyl, or 6-(N-(7-nitrobenz-2-oxa-1,3-diazol-4-yl)amino)hexanoyl; and
R₃ is H or methyl.

11. A method of treating pancreatitis or obesity in a subject in need thereof comprising administration of a therapeutically effective amount of a compound of formula (I): 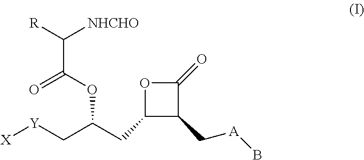
wherein,
R is (C₁-C₁₂)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is a solid support or H;
X is a solid support or H;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100; or a pharmaceutically acceptable salt thereof.

12. The method of claim 11, wherein
R is (C₁-C₆)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is H;
X is a solid support;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100.

13. The method of claim 11, wherein
R is (C₁-C₄)-alkyl;
A is (C₁-C₁₂)-alkyl;
B is H;
X is polyvinyl chloride or cellulose;
Y is a linking group comprising -J-, —O-J-, or —S-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
n is 0-10.

14. A device comprising
a) a compound of formula (I): 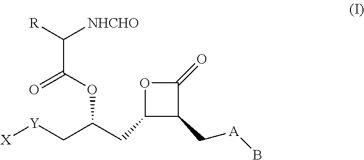
wherein,
R is (C₁-C₁₂)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is a solid support or H;
X is a solid support or H, wherein at least one of B or X is a solid support;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100; or a pharmaceutically acceptable salt thereof;
wherein the solid support comprises a material compatible to contact with blood;
b) a first conduit configured to deliver blood of a subject to contact the compound of formula (I); and
c) a second conduit configured to return blood to the subject.

15. The device of claim 14, wherein the solid support comprises a glass slide, a polymer bead, plastic tubing, glass tubing, rubber tubing.

16. The device of claim 15, wherein the solid support comprises medical grade polyvinyl chloride tubing.

17. The device of claim 14, wherein the first and second conduit comprise medical grade tubing.

18. A method of treating pancreatitis or obesity in a subject in need thereof comprising contacting the blood of the subject with a compound of formula (I): 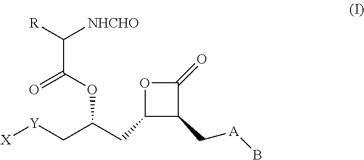
wherein,
R is (C₁-C₁₂)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is a solid support or H;
X is a solid support or H;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100; or a pharmaceutically acceptable salt thereof.

19. The method of claim 18, wherein
R is (C₁-C₆)-alkyl or (C₆-C₁₂)-aryl;
A is a linking group comprising —V—, —V—O—, —V—S—, —V—N(H)—, or —V—N((C₁-C₃)-alkyl)-;
V is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group;
B is H;
X is a solid support;
Y is a linking group comprising -J-, —O-J-, —S-J-, —N(H)-J-, or —N((C₁-C₃)-alkyl)-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
each n is independently 0-100.

20. The method of claim 18, wherein
R is (C₁-C₄)-alkyl;
A is (C₁-C₁₂)-alkyl;
B is H;
X is polyvinyl chloride or cellulose;
Y is a linking group comprising -J-, —O-J-, or —S-J-;
J is (C₁-C₁₂)-alkyl or —(OCH₂CH₂)ₙ—, wherein any carbon atom in said (C₁-C₁₂)-alkyl or —(OCH₂CH₂)ₙ— is optionally replaced with one or more heteroatom, cycloalkyl group, heterocycle, aryl or heteroaryl group; and
n is 0-10.

21. A method of treating pancreatitis or obesity comprising contacting the blood of a subject with a solid-supported inhibitor of lipase, or proteases, or phospholipase A2, or any combination thereof, and passing the blood of the patient over the solid-supported inhibitor with any device that then returns the blood to the patient.

22. A method of treating shock comprising contacting the blood of a subject with a solid-supported inhibitor of lipase, or proteases, or phospholipase A2, or any combination thereof, and passing the blood of the patient over the solid-supported inhibitor with any device that then returns the blood to the patient.

23. A method of treating pancreatic necrosis comprising contacting the blood of a subject with a solid-supported inhibitor of lipase, or proteases, or phospholipase A2, or any combination thereof, and passing the blood of the patient over the solid-supported inhibitor with any device that then returns the blood to the patient.

24. A method of assaying DAGL activity comprising contacting a compound with a compound of formula (II): 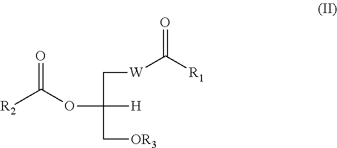
wherein,
W is O, NH, or N—(C₁-C₃)-alkyl;
R₁ is (C₁-C₁₂)-alkyl; (C₁-C₁₂)-alkyl-aryl, wherein aryl is optionally substituted with one or more nitro groups; (C₁-C₁₂)-alkyl-NH-aryl, wherein aryl is optionally substituted with one or more nitro groups; —NH(C₁-C₈)-alkyl, —O(C₁-C₈)-alkyl, —NH(C₁-C₈)-alkyl, 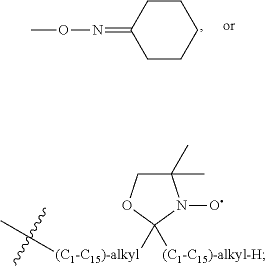
R₂ is (C₁-C₂₀)-alkyl; (C₁-C₂₀)-alkenyl; (C₁-C₂₀)-alkyl-aryl, wherein aryl is optionally substituted with one or more nitro groups; (C₁-C₂₀)-alkyl-NH-aryl, wherein aryl is optionally substituted with one or more nitro groups; (C₁-C₂₀)-alkyl-heteroaryl, wherein heteroaryl is optionally substituted with one or more nitro groups; or —NH(C₁-C₈)-alkyl; and
R₃ is H or (C₁-C₁₂)-alkyl; or a pharmaceutically acceptable salt thereof.

25. The method of claim 24, wherein
W is O, NH, or N—(C₁-C₃)-alkyl; 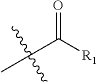
is 4-pyrenebutyryl, 10-pyrenedecanoyl, 5-doxylstearoyl, 16-doxylstearoyl, or dinitrophenyl-∈-amino-n-caproyl; 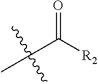
is 4-pyrenebutyryl, dinitrophenyl-∈-amino-n-caproyl, or 6-(N-(7-nitrobenz-2-oxa-1,3-diazol-4-yl)amino)hexanoyl; and
R₃ is H or (C₁-C₂)-alkyl.

26. The method of claim 24, wherein
W is O; 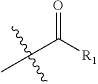
is 4-pyrenebutyryl, 10-pyrenedecanoyl, 5-doxylstearoyl, 16-doxylstearoyl, or dinitrophenyl-∈-amino-n-caproyl; 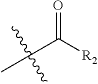
is 4-pyrenebutyryl, dinitrophenyl-∈-amino-n-caproyl, or 6-(N-(7-nitrobenz-2-oxa-1,3-diazol-4-yl)amino)hexanoyl; and
R₃ is H or methyl.
